(19) United States
(12) Patent Application Publication
(10) Pub. No.: US 2007/0053842 A1
(43) Pub. Date: Mar. 8, 2007

(54) Method of engineering particles for use in the delivery of drugs via inhalation

(76) Inventor: Joseph Okpala, Liverpool (GB)

Correspondence Address: DENNISON, SCHULTZ & MACDONALD, 1727 KING STREET, SUITE 105, ALEXANDRIA, VA, 22314, US

(21) Appl. No.: 10/582,473

(22) PCT Filed: Dec. 13, 2004
(86) PCT No.: PCT/GB04/05203

§ 371 Date: Jul. 3, 2006

(30) Foreign Application Priority Data
Dec. 12, 2003 (GB) 0328959.2
Mar. 22, 2004 (GB) 0406325.1

(51) Int. Cl. A61K 9/14; B29B 9/00

(52) U.S. Cl. 424/46; 264/5

(57) ABSTRACT

The present invention provides a method for engineering particles for use in the delivery of drugs via inhalation and involves the use of an artificial respiratory airway that simulates a mammalian lung system to create an environment in which particles can be engineered. The method utilizes the inhalation flow rate created within the artificial respiratory airway to optimize the particle attributes, engineered by the method, to create particles that are tailored to the specific inhalation flow rates of different patients.

### FIELD OF THE INVENTION

The present invention relates to the field of particle engineering, and more specifically to the engineering of particles for the use in the delivery of drugs via inhalation.

### BACKGROUND OF THE INVENTION

Different patients generate different inhalation flow rates depending upon their age, airway conditions and disease states. The current marketed inhaler products are standard i.e. intended for all patient categories irrespective of age and disease state. Patients with lower inhalation flow rate are forced to adapt themselves to the requirements of inhaled products. Unfortunately, this is unsuccessful for such patients, leading to patients exacerbation, sub-optimal therapeutic dose (and effect), increased side effects, medicine wastage and ultimately to non-compliance by the patient.

As stated in U.S. Pat. No. 6,060,069, “Inspiratory flow rate is the air velocity a patient generates when inhaling. In healthy adults, during tidal breathing, inspiratory flow rate is about 15 L/min, and with effort, inspiratory flow rates of 100 L/min or more are easily achievable. Inspiratory flow rate in adult patients with moderate to severe obstructive airways diseases has been demonstrated to average 25.4 L/min (ranging from 13.3 to 50.4 L/min). Asthma is an obstructive airways disease, it may have a dramatic effect on the ability of adults and children with asthma to create an inspiratory flow rate adequate to operate most DPI's currently available”.

Hence for those patients who are unable to generate sufficient inspiratory flow rates (such as patient with chronic obstructive airways diseases, young children, the elderly and those with acute or chronic respiratory conditions) to activate and operate the DPI's will result in the majority of drug depositing in the throat and upper airways, where it provides little or no therapeutic benefit and may cause side-effects, when swallowed or absorbed from their site of deposition.

From the above it is obvious that the patient inhalation flow rate is paramount in determining drug deposition into the lungs, unfortunately, conventional inhaled products have no means to assure that the drug particles, in the final inhaled product are at least suited to any patient inhalation flow rate before producing the final inhaled product. The conventional procedures adopted in inhalation science are:
producing inhaled drug particles by milling (such as micronisation) or any other techniques know in the art.
particle size measurement of the produced drug particles by laser diffraction, electrical zone sensing or any other sizing techniques to ensure that the drug particles are within the geometric size range 1-5 μm. If the particles do not comply with this size range, the particles are reprocessed in order to try and achieve this size requirement. Where the particle size requirement is achieved there is no guarantee that these particles are favourable for deep lung penetration especially as the drawbacks associated with the production of fine inhaled drug particles as listed in the prior art (some of which are the amorphous content, oversize and undersize etc), prevent adequate deep lung penetration of inhaled drug particles.
The resulting drug particles are then formulated for Dry Powder Inhalers (DPI's) or Metered Dose Inhalers (MDI's) and then tested, in vitro, using any suitable inertial impaction technique (for example, Twin stage impiner, Cascade Impactor, five stage liquid impinger and the like), at different inhalation flow rates, to determine the deposition profile of the resulting particles. The powders collected from the stages of the inertial impactor are dissolved in an appropriate solvent to enable quantification of the mass on each stage using an appropriate analytical method. The formulating, testing (at different inhalation flow rates) and analyses are time consuming and uneconomic and still does not guarantee adequate deep lung deposition at any inhalation flow rate.

Conventional teaching and practise for producing particles for inhalation is unable, to date, to take into consideration the inhalation effort of the patient, at the time of production, to produce aerodynamically favourable particles to suit that patient and/or the disease state whilst giving high amount of drug depositing in the targeted area of the lung and especially deep lung.

Conventional DPI's and MDI's are standard formulations intended for all categories of patients and their disease states irrespective of their inhalation flow rate generating capabilities. Studies in the literature have shown that the amount of drug depositing to the lower airways increased with patient inhalation flow rate. Hence patients with the highest flow rates obtain better therapeutic effect whilst patients with the lowest inhalation flow rates would probably receive minimum if not sub-optimal therapeutic effect. From this, it can be concluded that the conventional, standard DPI's and MDI's are inappropriate and inapplicable for use in all patient categories as they are not independent of patient inhalation flow rate. Furthermore, conventional DPI's, MDI's and inhalation practises forces the patient to adapt (i.e. to increase) their inhalation flow rate to try and achieve therapeutic benefit from the standard formulations. Unfortunately, patients whose maximum inhalation flow rate is restricted would not benefit from the use of these standard formulations.

From the limitations of conventional and current inhalation practises, there is a need to develop new inhalation particles or formulations which are adaptable and suitable for a specific patient or for a broad range of patients in terms of their inhalation flow rates and the inhaled product containing these particles should be suitably labelled to indicate the inhalation flow rate used to generate them and/or the specific and or range of patient inhalation flow rate who would obtain maximum benefit from the product. Where the product is inhalation flow rate independent or inhalation flow rate dependent it should also be labelled accordingly. Such suitably labelled inhaled products hastens and simplifies the correct choice by the patient and health care professionals of the product that best suits the patient inhalation flow rate at that time.

### PRIOR ART

A discussion of relevant prior art in the technology field of particle engineering is provided below. Techniques known in the art to obtain particles of 1-5 μm are milling, crystallisation, spray-drying, spray freeze drying and supercritical fluid to name a few common examples. However, these techniques are fraught with problems some of which are briefly described below.

The milling process is undesirable for several reasons in that it has the potential to change more than the particle size of the feed material. The heat generated during inter-particle collisions causes changes in the solid state thereby affecting the crystallinity and stability of the milled material. Milling dramatically reduces the crystallinity of the feed material as reported, for example, with albuterol sulphate (Ward, G. H. and Schultz, R. K. [1995]. Process-induced crystallinity changes in albuterol sulphate and its effect on powder physical stability. Pharm. Res. 12, 773-779) and microcrystalline cellulose (Ogura, K. and Sobue, H. (1970) Changes in morphology with milling of the commercial microcrystalline cellulose. J. App. Polymer Sci. 14, 1390-1393). For the latter, reductions of crystallinity of at least 23% was observed. Even synthetic polymeric macromolecules such as polyvinylpyrrolidone were damaged by milling in that it's molecular weight decreased during milling (Kaneniwa, N. and Ikekawa, A., 1972, Influence of ball-milling atmosphere on decrease of molecular weight of polyvinylpyrrolidone powders. Chem. Pharm. Bull. 20, 1536-154), this is extremely relevant to the milling of biomolecule powders. Further, chemical decomposition of thermally labile molecules has been observed during micronisation. Furthermore, brittle materials will tend to fracture (as fracturing is a requirement for successful milling) during inter-particle collisions while ductile materials will tend to undergo plastic deformation and change shape rather than fracture (Van Vlack, L. H. (1980). Elements of materials science and engineering. Addison Wesley, Publishing, Reading, M A, pp. 185-208). Additionally, the milled powder is highly cohesive, forming agglomerates that are very difficult to mix due to poor and incomplete dispersion of agglomerates into their single particles. Milling, also, generates a significant fraction of unwanted under-size particles. The under-size material, if it remains, is considered wasted and should preferably be removed, thus making the milling process uneconomic. Other important aspects are that milling does not give the user control over particle attributes such as, the aerodynamic properties of the particle, particle density, particle shape and particle surface texture. Furthermore, the milling process exposes the personnel to the hazardous effect of the fine dust coupled with high product loss. Additionally, milling is only applicable for substances in the dry state or particles suspended in a liquid and cannot operate over a wide temperature range.

Conditioning has been reported in the literature (Patent WO 95/05805; U.S. Pat. No. 5,562,923; U.S. Pat. No. 5,874,063) as a method to remove amorphous content that may have been generated by a milling process. Conditioning, as reported in these patents involved treatment of fine dried material with a solvent in the vapour phase, without affecting the aerodynamic properties and other specifications of the particle (for example particle size, particle shape, presence of oversize and under-size particles, presence of particle agglomerates, irregularities in particle shape and surface texture such as sharp edges etc) by this treatment. The authors of the patents WO 95/05805 and U.S. Pat. No. 5,874,063 provided no details of any changes in surface properties or shape of conditioned particles. Thus, if the particles before conditioning contain any undesirable properties, such as for example undersize or oversize particles or presence of crevices, asperities, sharp edges or clefts, these will still remain in the final conditioned particles. As a result the conditioned powder is still not optimal for use, but will instead need further treatments to obtain powder with the desired properties such as shape for example in which case a further treatment step of spheronisation, as described in U.S. Pat. No. 6,287,540 or U.S. Pat. No. 5,551,489 was used. Further, conditioning the powder using water or organic solvent vapour is a time consuming process taking up to 100 hours or more. Additionally, the powder bed that is being conditioned is usually static or inverted from time to time, as a result all the powder is not contacted with the vapour to the same amount, to the same extent nor in an uniform manner and thus it is also an inefficient process. The end point of conditioning is based on “guess estimation”, thus any excess solvent may cause clumping between particles thereby increasing the particle size and size range. Importantly, conditioning “probably rearranges the outer surface layer of the crystals of the amorphous substance” (U.S. Pat. No. 5,562,923) whilst the interior of the particles remain amorphous. Consequently, the final conditioned particles are partially crystalline and thus unstable and will revert to the more stable form depending on the storage conditions.

US Patent 2002/0168395 A1 is another conditioning technique and describes a process for effecting a solid-state crystallization of spherical shaped microspheres produced by spray congealing. The microspheres were crystallised in an atmosphere saturated with the vapours of a solvent or non-solvent. The recovered microspheres were crystalline, but retained their size and shape after treatment. The authors gave no examples of particle size before and after treatment. Again, this technique still suffers from the draw-backs of the conditioning techniques described above.

In order to alleviate the long treatment times and potential particle fusion problems associated with conditioning in patents such as WO 95/05805, Patent WO 99/34778 discloses a process of stabilising powders using wet suspension. The particles are suspended in a suspending agent, followed by evaporation of suspending agent to harvest the particles. This technique has many drawbacks, one of which is the toxicity of the suspending agent, such as n-heptane and n-hexane used in WO 99/34778. WO 99/34778 also mentioned the use of N-alkanes such as methanol, ethanol, acetone and the like. These N-alkanes, are known to be non-solvents for the drugs and carriers (lactose) mentioned in WO 99/34778 as a result crystal growth of some treated substances might occur in N-alkanes, changing not only the particle shape but also their size and size distribution. These effects are accentuated especially if heat and long evaporation times were involved as was the case in Patent WO 99/34778. Immersion of the particles in a suspending agent may ensure efficient and uniform contact between the particles and reduce the treatment time but it does not alleviate the particle fusion problems. Further-more there is the difficulty in harvesting small particles from a suspending medium.

U.S. Pat. No. 6,132,797 describes a conditioning process for producing sugar crystallites ‘for comestibles’ by contacting amorphous sugar with a liquid non-solvent. The polycrystallite sugar are not greater than 200 micrometer, their disintegration in sugar-saturated aqueous liquid provide crystallites with an average size of 10 micrometer or less. The author provided no details for disintegrating the polycrystallite, nor harvesting the crystallites from sugar-saturated liquid solution.

From the above, it is clear that micronising produces particles that have at least 4 disadvantages which are; aggregation, amorphous content, under and oversize, irregular and non-uniform shape. It should also be equally clear that, irrespective of the conditioning technique used, conditioning attempts only to alleviate one of these disadvantages i.e reducing the amorphous content in which case the particles still retain the remaining disadvantages. From this it is understood to those skilled in the art, that conditioning is not necessarily the best option to obtain powder with optimal physico-chemical characteristics. Another disadvantage shared by all the above mentioned patents are that all the particles remain in contact with each other at all times during the conditioning and can therefore interfere with each other. Where conditioning is performed in a liquid, harvesting conditioned particles from a liquid medium, especially where the particles are small, is a challenge in itself even without considering the problems associated with particle fusion from such treatments. Then there are further problems associated with the correct choice of liquid medium as to whether the particles are soluble or insoluble in the liquid medium causing particle growth or whether the medium affects the stability of the constituents of the particle.

The crystallisation process requires considerable time and energy resources and defines such economical issues as efficiency of solvent recycling, separation of waste (impurities) and consumption of raw materials. It is acknowledged that minor changes in crystallisation conditions, for example super-saturation, temperature, impurity or cooling rate can produce significant changes in the crystal and powder properties notably, particle size, shape, purity and defect structure followed by less pronounced but significant variations in thermodynamic and mechanical properties. These effects have been recognised as the major batch to batch and source variation problems leading to inconsistencies of the final product. Crystals with uniform shape and desired size range especially between 1-5 μm are extremely difficult to achieve by crystallisation from solution as in most cases the particles grow too fast to be recovered from the liquid medium. Further, even in cases where such particle sizes can be formed and maintained, the recovery of individual un-agglomerated particles of this size from liquid is near impossible. Hence milling is then the preferred option to obtain particles of 1-5 μm size range, despite all the disadvantages of micronisation as described above.

There are many examples of modified crystallisation from solution processes that facilitate the recovery of the formed particles one of which is U.S. Pat. No. 6,221,398. This patent describes a process for producing a pharmaceutical powder for inhalation comprising dissolving an inhalation compound in a solvent and introducing the solution containing the inhalation compound in droplet form or as a jet stream into an anti-solvent which is miscible with the solvent and which is under agitation. The particles produced were of 10 micrometer or less, however, the authors of this patents provided no details of the particle shape. To those skilled in the art the shape of the inhaled particles is important for successful inhalation therapy.

Another modified crystallisation from solution process is U.S. Pat. No. 6,074,441, which describes a process for producing ultrafine-crystallisation products ‘for non-inhalation purposes’ with an average particle diameter of <1 micrometer. The process is based on atomisation of a solution and simultaneous evaporation of the solvent to form a crystalline product. The crystallisation occurred in a gas atmosphere and the particles obtained are too small (<1 μm) to be effectively retained in the lungs during inhalation.

Patent WO 94/07582 describes a dual jet crystalliser apparatus for direct and immediate crystallisation of pharmaceutical and chemical compounds. Finasteride, acetic acid and water was instantly crystallised by the dual-jet. The average particle size of the recovered crystalline product was 10-15 um in the form of hexagonal flakes. It is understood to those skilled in the art that the crystals produced are too large to be used for inhalation.

From the above, it is obvious that with crystallisation from solution it is difficult to form and recover particles in the size range 1-5 μm that are not agglomerated, that are narrow in size and size distribution with high degree of particle uniformity. Furthermore, crystallisation is time and energy consuming and that minor changes in crystallisation conditions causes significant batch to batch and source variation problems leading to inconsistencies of the final product. Additionally, agglomerates, under size and oversize particles are obtained with the crystallisation process. Hence crystallisation can be used to alleviate one major disadvantages of milling which is amorphousness but crystallisation has difficulty resolving issues relating to crystal consistency as regards to particle shape, surface texture and under and oversize particles.

Spray drying has been seen as an alternative technique to micronisation as the shape of the particle is nearly always spherical whilst producing particles with a narrow size distribution. However, the material formed contains various degrees of amorphous regions. Such regions are often more sensitive to external conditions e.g. moisture, thus making the particles more susceptible to chemical degradation. The relatively high temperatures required to dry the droplets may facilitate product degradation making spray drying unsuitable for thermo-labile substances. Furthermore, the particles produced are always cohesive and have poor flow and hence cannot be realistically aerosolised (Kawashima, Y et al., (1998), Effect of surface morphology of carrier lactose on dry powder inhalation property of pranlukast hydrate, International Journal of Pharmaceutics, 172, 179-188). Additionally, the recovery of the material is poor as a substantial quantity of the material is vented and thus wasted, especially those particles that are very fine, thus making spray drying uneconomic for expensive drugs. This technique is only applicable to substances present in the liquid state and can only be used to process materials above ambient temperatures. Spray drying has difficulties processing hydrophobic materials as these substances need to be dissolved in organic liquid before spray drying. These organic liquids are invariably explosive at the temperatures required to effectively spray dry.

Patent WO 02/28377 A1 discloses a process of forming rough spherical particles by a modified spray drying process in which the liquid feed stock is atomised into a heated column at temperatures below 300° C. The resulting particles were collected using a 16 kV corona discharge arc and subsequently scraped from the discharge plate. This technique is only applicable to substances present in a liquid feed stock, the high voltage used will damage fragile materials, temperatures below 300° C. are claimed, however, in reality a temperature high enough to prevent re-condensation of the liquid of the feed stock is necessary and consequently results in the use of temperatures above 70° C. Additionally, since it is necessary to scrape the particles from the discharge plate this suggests that the particles are cohesive.

Another example of a modified spray drying process is U.S. Pat. No. 6,051,257, in which an atomised liquid droplets of a liquid feed stock passed through an impactor to classify and remove droplets greater than a pre-determined size subsequently followed by passing said droplets through an elongated heating zone at a temperature from about 100° C. to 300° C. to dry the droplets and form spherical solid particles. This process has many limitations some of which are listed here; only liquid solutions can be used, over-size droplets are removed but under-size droplets remain, rapid drying caused by the use of high temperatures will generate amorphous nature in the dry particle, such high temperatures are unsuitable for thermo-labile and fragile materials as claimed in that patent, fusion of liquid droplets post classification will form oversize or agglomerated particles that remain in the final product and positively skews the size distribution towards the larger size and the particle shape is limited to spheres or spheroidal.

All the techniques relating to spray drying such as that described in U.S. Pat. No. 6,074,441, U.S. Pat. No. 5,314,506, Patent WO 02/28377 A1, U.S. Pat. No. 6,051,257 are limited to liquid feeds stocks. Further, the shape of the final particle is usually the same as the original droplet i.e spherical. The relatively high temperatures used to dry the particles are unsuitable for thermo-labile substances. Additionally, the particles produced are highly amorphous and thus cohesive coupled with high product loss.

Of all the above processes micronising is still the predominant process for forming inhaled products despite all of the disadvantages of milling as detailed above. In conventional inhalation practise, there is no prior knowledge of the inhalation characteristics of the particles used to form the inhaled product. The particles are only tested for their inhaled deposition profiles only after both post-production and post formulation. Most importantly, it is known that the inspiratory capacity of the end user, i.e the patient, is paramount in determining the success of the inhalation dosage form. Unfortunately, the patient is only considered as a last resort at the very end of the manufacturing process. Consequently, the formulation may be useful for some patients but more importantly it will not be useful for the frequent users of the inhaled dosage forms who can only generate low inspiratory, inhalation flow rates. The common practise is to manufacture the same powder in different doses for different patients according to their age and disease state. However, if an adult with a mild airway condition, who can generate high inspiratory flow rates, cannot obtain the full dose from the inhalation dosage form. Thus how would children, the elderly and patients with severe airway conditions, who can only generate low inspiratory flow rates, be expected to get the full dose into the lung?. From this it is understood that the same formulations cannot be adapted to all categories of patients and thus it is therefore necessary to review common practises. Ideally it is preferred that each inhaled dosage form should be adapted to any particular patient, unfortunately, in reality, this would be uneconomic, difficult and too expensive to achieve as there is a large number of patient categories and disease states and more importantly, there are no particle engineering processes available that enables the attainment of particles that achieve the above goals. The preferred and economic remedy is an engineered formulation that is independent of patient inspiratory flow rate or engineering particles specifically matched to low inspiratory flow rates that give high deposition of these particles to deep lung. Hence patients that can generate much higher inspiration flow rates will have no difficulty obtaining the full dose. Ideally the inhaled particles intended for deep lung penetration should be engineered under the test conditions i.e engineering at flow rates matching patient inspiratory flow rate whilst ensuring high deposition profile of the engineered particles. The engineered particles should be capable of being engineered from any state of matter (vapour, liquid, solid and frozen states of matter) irrespective of the physico-chemical properties (such as hydrophilic, hydrophobic and combinations of substances). The particles produced should have a high degree of mono-dispersity to enable targeting of the particles to the desired region of the airways. The engineering process should be flexible, to work over a wide range of flow rates to match all categories of patients and their disease states. The engineering process should enable the operator to add further particle attributes and modify or manipulate existing particle attributes, in situations where the patient inhalation flow rate, on its own, is incapable of producing, in order to improve the delivery capabilities of the resulting particles. The process should not be limited by the temperature range but should be able, for example, to operate over a wide temperature range at negative, and positive temperatures. Hence, thermo-resistant and thermo-labile substances can easily be engineered without affecting their physico-chemical stability and biological activity. The engineering process should be simple, cost-effective, scalable to meet market requirements and free from the drawbacks of the prior art. These are some of the objectives of the present invention.

### SUMMARY OF THE INVENTION

A method of producing particles for the use in the delivery of drugs by inhalation, whereby the attributes of the particles are engineered to fit the needs of a selected patient type, said method comprising the steps of:

a) providing an artificial respiratory system, which stimulates at least one of the drug delivery target regions of the mammalian respiratory system;

b) operating the artificial respiratory system to simulate a controlled inhalation flow rate with the system;

c) introducing a feedstock material into the artificial respiratory system, whereby said feedstock material provides a main constituent of the particles to be engineered;

d) creating an environment within the artificial respiratory system that is conducive to the production of engineered particles from the feedstock material; and

e) collecting the resultant engineered particles from at least one of the simulated drug delivery target regions provided by the artificial respiratory system.

The strategy of the present invention is to use patient inhalation flow rate to engineer the inhaled products as it is more likely that this inhalation flow rate would engineer a product with attributes and consequently aerodynamic properties that are at least, suited for that inhalation flow rate. Since the patient inhalation flow rate is used to engineer and/or produce the particles, it is more likely that the resultant particles would suit that patient inhalation flow rate to give the intended therapeutic effect without exacerbating the patient.

Furthermore, by engineering particles and testing the particles in a mimicked respiratory system to ensure maximum particle deposition in the required region(s) of the mimicked respiratory system, it is possible to target any desired region within the airways for any patient inhalation flow rate.

Additionally, the strategy should enable the engineering of particles in a mimicked respiratory airway containing mimicked respiratory fluid(s) to produce particles that are stable to the airways environment of the end user whilst limiting the effects of airways environments on the deposition profiles of the particles. The strategy should also facilitate the engineering of particles with a wide variety of attributes that suit the drug, patient inhalation flow rate and target region(s) of the respiratory airway.

This approach, contrary to the prior art, where the patient is only considered post-production, takes into account the patient inhalation flow rate at beginning and right throughout the entire engineering and testing process. There are no other techniques, available in the prior art, which can engineer particles in conditions mimicking the airways to ensure that the engineered particles can specifically target one or more region(s) whilst ensuring high deposition of the engineered particles to the targeted region(s). Thus this invention emphasises the importance of bringing the engineering process more in line with the requirements of the end user to facilitate drug targeting and delivery in order to maximise therapeutic effect whilst minimising patient exacerbation and side-effects.

The patient is extremely important as they are the end user of the product, hence, the particles must be produced to suit their inhalation flow rate and disease state to achieve the intended therapeutic effect.

Since the particles are engineered and/or produced by the action of the inhalation gas flow rate matching that of the patient,
1 Less training by health care professionals to the patients is required.
2 Lower likelihood to exacerbate the patient by forcing them to generate the high inhalation flow rates demanded by the prior art. The likely users of the inhaled products are known to be incapable of generating such high inhalation flow rates.
3 The patient with a knowledge of their inhalation flow rate will be more confident in obtaining symptoms relief even before trying the inhaler. Thus increasing patient compliance and consequently increasing the efficiency of therapy.
4 Knowledge of the patient inhalation flow rate and product identification showing the inhalation flow rate range over which the formulation is operable enables appropriate selection of the inhaler device by health care professionals and the patient to suit patient requirement at the time of use of inhaler.

Notification of the inhalation flow rate range on the inhaler device is paramount as it will help the identification (by health care professionals and the patients) and selection of the appropriate inhaler device without confusion and the associated waste of time.

The present invention adopts the principles of effervescence on its own or in conjunction with the patient inhalation flow rate to prepare inhaled products which have morphological and/or topographical attributes that make them favourable for inhalation therapy.

To enable the inhalation flow rate to efficiently engineer the inhaled products with attributes that make them aerodynamically favourable, the invention adopts the following strategies:
a) De-aggregate and disperse the product to form aerosol particles that are dispersed in a gas
b) Use the inhalation flow rate to further de-aggregate and disperse the aerosol particles to increase the specific surface area of the aerosol particles whilst carrying it through an inhalation flow zone that comprises at least a mimicked respiratory system and optionally one or more spacer devices.
c) Use the inhalation flow rate not only to carry the aerosol particles but also to carry and increase the specific surface area of a preformed engineering environment and/or to generate, carry and increase the specific surface area of engineering environments formed by vaporisation of liquid(s) in the inhalation flow zone, which are used to dynamically humidify the inhaled gas and promote mixing of the vapour-loaded and aerosol particle loaded inhaled gas to form microenvironments containing a finite amount of the vapour, around each aerosol particle, that is used to moisturise that aerosol particle,
d) Using the inhalation power, which is a combination of the inhalation flow rate and the time over which this inhalation flow rate acts upon the aerosol particles, to force the transfer of moisture in each microenvironment onto, into and out of the associated aerosol particle at rates and to extents controlled by the inhalation flow rate to engineer and form particles whose morphology, topography and aerodynamic properties have been dictated by the inhalation flow rate.
e) Use the inhalation flow rate to additionally keep the aerosol particles away from each other during engineering and in flight so as to minimise aggregation, sticking and fusion of the aerosol particles.

These strategies of the present invention enabled particles to be engineered, in flight, in a uniform manner that will consequently give a final product that is uniform, mono-disperse and of defined shape, forms and dimensions, that, importantly, have aerodynamic properties favourable, at least, for the inhalation flow rate used to engineer them. The strategy further enables the particles deposited on a solid surface or in a liquid medium to be further engineered by the action of the inhalation flow rate.

The systems and processes of the invention enable aerodynamically favourable inhalable particles to be engineered from a variety of feedstock including feedstocks which are in the solid state, liquid state, frozen state, vapour state and combinations thereof. The process is able to use any delivery devices that can aerosolise the foregoing to enable the inhalation flow rate to act on each individual aerosol particle. Such devices can be breath-actuated i.e. using the inhalation flow rate to de-aggregate and disperse the aerosol particles or self-actuated i.e. the power of the device to de-aggregate, disperse and aerosolise the particles. The feedstock can comprise one or more substances, which may or may not be therapeutically active.

In one embodiment of the current invention, the inhalation flow rate uses liquids present in the human lungs and the temperature within the lungs to engineer particles that maximally deposit in targeted regions of the airways whilst stabilising these particles to the liquids present in the lungs. This produces aerodynamically favourable particles that are consequently quality assured for the environment within the patient airways.

In a preferred aspect of the present invention, the inhalation power dictates particle attributes. In another aspect of the present invention the inhalation gas flow rate dictates particle attributes. These attributes can be but are not limited to morphology, topography and aerodynamic properties to give particles that are more favourable for at least that inhalation flow rate. Suitable morphological attributes can be but are not limited to particle size, shape, volume and surface area. Suitable shapes include but are not limited to cubes, cuboidal, spheres, hemi-spheres and fibre-like shapes and combinations thereof. Suitable particle size can range from at least 0.5 μm to 80 μm. Suitable topographies include but are not limited to controlling surface regularity either by increasing surface smoothness, increasing surface roughness such as but not limited to the formation of “hairs”, causing depressions, golf ball like particles, dents, indentations, cat's tongue, ridges, sharkskin, striae, channels, entrances and exit into and out of the particles. Additionally, the process and system of the present invention enables the aerodynamic diameter to be controlled to an accuracy of at least 0.3 μm.

In a preferred aspect of the present invention, the topography of the particles can be controlled without affecting the morphology of the particles. In a preferred aspect of the present invention, the morphology of the particles can be controlled without affecting the topography of the particles. In a further preferred aspect of the present invention, the morphology and topography of the particles can both be controlled.

The morphological and topographical attributes of the engineered particles can give these particles high specific surface area and this is suitable for hydrophobic drugs whose dissolution is rate-limiting step for their absorption.

Another aspect of the present invention is, for the same drug, the same patient inhalation flow rate and the same the same target region(s) in the respiratory airways, enables the engineering of particles with a wide variety of attributes that suit these requirements. This has important implications as it enables the selection of particle attributes that maximises therapeutic effect whilst reducing the dose required for the therapeutic effect. This is beneficial to the patient, as there are less side effects, and to the manufacturer as lower doses are used and maximum therapeutic effect is achieved thus enabling cost savings.

In another embodiment of the present invention, effervescence is used alone or in conjunction with the inhalation flow rate to engineer particles with topographical, morphological and aerodynamic properties that make them favourable for inhalation therapy. Effervescent formulations are also desirable for delivery to the lungs in that they produce bubbles which are aerodynamically favourable and are carried and directed by the inhalation flow rate to deep lung. The high gaseous contents of the bubbles enables rapid diffusion of the bubble contents into and through the lung fluids and lung membranes. Additionally the particles are made more aerodynamically favourable to the inhalation flow rate by effervescence, hence more particles should reach and deposit in the lower airways. Those particles that deposit in the lower airways will further effervesce in the lung fluids causing particle disintegration, increasing the specific surface area of the deposited particles from which there is improved drug dissolution and bioavailability. Consequently, effervescent particles are applicable for delivery to upper and lower airways to give local and/or systemic effect.

The philosophy of the current invention and the processing systems of the current invention preferably require the engineered particles, formulations containing the engineered particles and compositions and/or inhaler devices containing the engineered particles to be suitably identified in the patient information leaflet and/or the inhaler device to indicate the inhalation flow rate used to engineer and test the particles and also indicating the patient inhalation flow rate which the engineered particles give maximum targeted deposition as well as indicating the regions of the lung targeted by these engineered particles at any inhalation flow rates. Hence, by measuring patient inhalation flow rate using devices such as peak flow meters, it is possible to give the patient the appropriate inhaler device containing engineered particles that maximally deposit at the target regions of the lung required by the patient at that patient's inhalation flow rate. This consequently enables easy identification by the patient or healthcare professional of the formulation compositions or inhaler device containing the foregoing that best suits the patient according to their patient inhalation flow rate. This should be applied to all inhaler devices not just those containing engineered particles of the current invention.

### BRIEF DESCRIPTION OF THE DRAWINGS

The method the present invention and particles produced by the method will be described hereinafter with reference to the drawings, which comprise:

FIG. 1 shows a schematic representation of the process in accordance with the present invention;

FIG. 2 shows a scanning electron micrograph of smooth spray dried lactose;

FIG. 3-1 shows a standard twin stage impinger (TSI) of Apparatus A as described in the British Pharmacopoeia, BP 2003;

FIG. 3-2 shows a mTSI showing the arrangement of the coupling tube and microscope stub;

FIG. 4 shows a general view, scanning electron micrograph of the engineered cubic lactose particles according to example 1;

FIG. 4.1 shows a close view, scanning electron micrograph of the engineered cubic lactose particles according to example 1;

FIG. 5 shows a close view, scanning electron micrograph of engineered cubic lactose particles according to example 2;

FIG. 6 shows a close view, scanning electron micrograph of the engineered cubic lactose particles;

FIG. 7 shows a scanning electron micrograph of a rough spherical lactose particle before engineering;

FIG. 8 shows a scanning electron micrograph of engineered cubic lactose particles according to example 4;

FIG. 9 shows a close view, scanning electron micrograph of engineered elegant salbutamol sulphate cubes;

FIG. 10 shows a schematic diagram used to spray freeze fracture aerosol particles from liquid feedstock that is then engineered by the inhalation gas flow;

FIG. 11 shows a scanning electron micrograph of a general view of BDP particles engineered according to example 6;

FIG. 12 shows a close view, scanning electron micrograph of a BDP particle engineered according to example 6;

FIG. 13 shows a schematic diagram spray freeze fracturing aerosol particles from liquid feedstock according to example 7;

FIG. 14 shows a scanning electron micrograph of a Beclomethasone dipropionate particle engineered according to example 7;

FIG. 15 shows a schematic diagram of the equipment used in example 8;

FIG. 16 shows a background view, scanning electron micrograph of Beclomethasone dipropionate particles obtained by example 8;

FIG. 17 shows a background view, scanning electron micrograph of Beclomethasone dipropionate particles obtained according to example 9;

FIG. 18 shows a schematic diagram describing the set-up used according to Example 10;

FIG. 19 shows a close view, scanning electron micrograph of Fluticasone propionate particles obtained according to example 10 at zero inhalation gas flow rate;

FIG. 20 shows a background view, scanning electron micrograph of Fluticasone propionate particles according to example 10 at an inhalation gas flow rate of 60 L/min;

FIG. 21 shows a background view, scanning electron micrograph of Fluticasone propionate particles engineered at an inhalation gas flow rate of 120 L/min according to example 10;

FIG. 22 shows a photograph of the assembly of the equipment used to engineer Fluticasone propionate particles at 28.3 and 60 L/min in an Andersen cascade impactor;

FIG. 23 shows a scanning electron micrograph of Fluticasone propionate particles engineered at inhalation flow rate of 28.3 L/min at ambient temperature according to example 11;

FIG. 24 shows a photograph of the in vitro deposition of engineered Fluticasone propionate particles on the Andersen cascade impactor plates at an inhalation flow rate of 28.3 L/min, at ambient temperature, according to example 11;

FIG. 25 shows a scanning electron micrograph of Fluticasone propionate particles engineered at inhalation flow rate of 60 L/min at ambient temperature according to example 11;

FIG. 26 shows a photograph of the in vitro deposition of engineered Fluticasone propionate particles on the Andersen cascade Impactor plates at an inhalation flow rate of 60 L/min, at ambient temperature according to example 11;

FIG. 27 shows a scanning electron micrograph of Fluticasone propionate particles engineered, at the temperature and humidity conditions found in the airways, at an inhalation flow rate of 28.3 L/min according to example 12;

FIG. 28 shows a photograph of the in vitro deposition of Fluticasone propionate particles on the Andersen cascade Impactor plates, engineered at the temperature and humidity conditions found in the airways at inhalation flow rate of 28.3 L/min according to example 12;

FIG. 29 shows a scanning electron micrograph of Fluticasone propionate particles engineered, at the temperature and humidity conditions found in the airways, at inhalation flow rate of 60 L/min according to example 12;

FIG. 30 shows a photograph of the in vitro deposition of Fluticasone propionate particles on the Andersen cascade Impactor plates, engineered at the temperature and humidity conditions found in the airways at inhalation flow rate of 60 L/min according to example 12;

FIG. 31 shows a scanning electron micrograph of Fluticasone propionate particles engineered at temperatures mimicking those in the human respiratory airway at inhalation flow rate of 28.3 L/min according to example 13;

FIG. 32 shows a background view, scanning electron micrograph of salbutamol sulphate particles engineered at an inhalation flow rate of 60 L/min and using a temperature of 45° C. inside an Andersen cascade impactor;

FIG. 33 shows a background view, scanning electron micrograph of salbutamol sulphate particles engineered at an inhalation flow rate of 120 L/min and using a temperature of 45° C. inside the mTSI;

FIG. 34 shows a schematic diagram describing the set-up used according to Example 17;

FIG. 35 shows a background view, scanning electron micrograph of bovine serum albumin particles engineered at an inhalation flow rate of 120 L/min at a temperature of 45° C. according to example 18;

FIG. 36 shows a background view, scanning electron micrograph of Beclomethasone dipropionate particles obtained at inhalation flow rate of 60 L/min according to example 19;

FIG. 37 shows a background view, scanning electron micrograph of a 10 to 1 ratio of Fluticasone propionate/Salmeterol xinafoate particles engineered at an inhalation flow rate of 60 L/min according to example 21;

FIG. 38 shows a close view, scanning electron micrograph of a 10 to 1 ratio of Fluticasone propionate/Salmeterol xinafoate particles engineered at an inhalation flow rate of 60 L/min according to example 21;

FIG. 39 shows a background view, scanning electron micrograph of Beclomethasone dipropionate/Salbutamol sulphate particles in the ratio 50:50 w/w according to example 22;

FIG. 40 shows a close view, scanning electron micrograph of Beclomethasone dipropionate/Salbutamol sulphate particles in the ratio 50:50 w/w according to example 22;

FIG. 41 shows a close view, scanning electron micrograph of Beclomethasone dipropionate/Salbutamol sulphate particles in the ratio 80/20 w/w according to Example 22;

FIG. 42 shows a schematic diagram describing the set-up used according to Example 23;

FIG. 43 shows a background view of lactose particles, engineered, by the inhalation gas flow rate, after deposition according to Example 23;

FIG. 44 shows a close view, of lactose particles, engineered, by the inhalation gas flow rate, after deposition according to Example 23;

FIG. 45 shows a schematic diagram describing the set-up used according to Example 24;

FIG. 46 shows a background view of Fluticasone Propionate particles, engineered by suction of FP solution from the airbrush into the spacer device and twin stage impinger;

FIG. 47 shows a schematic diagram describing the set-up used according to Example 25.

### DETAILED DESCRIPTION OF THE PRESENT INVENTION

Unless otherwise defined, all technical and scientific terms used herein have the same meaning as commonly understood by one skilled in the art to which this invention belongs. Although any methods and materials similar or equivalent to those described herein can be used, the preferred methods and materials are now described.

The aforementioned shortcomings and disadvantages of the prior art are overcome by the processes and particles resulting therefrom, provided by the present invention.

The present invention exists in more than one embodiment. The first embodiment of the present invention describes processes and systems that uses inhalation gas flow rate to engineer particles. The method of the present invention uses inhalation gas flow rate to engineer particles by conferring or dictating to the particles attributes that make these particles aerodynamically favourable for that inhalation flow rate. The term “inhalation gas flow rate” as used herein means the volume or amount of gas passing a given point per unit time wherein the unit time is measured in minutes. The terms “inhalation gas flow” and “inhalation gas flow rate” can be used interchangeably according to whichever befits the sentence in which it is placed. The term “engineer” is used interchangeably with the term “produce” to indicate the formation of particles whose attributes have been produced and/or modified by the action of the inhalation gas flow rate.

A second embodiment of the present invention relates to the application of the methods of the invention, that uses inhalation flow rates matched with patient inhalation flow rates to engineer and test (in-vitro in a mimicked airways) the particles whilst ensuring high drug deposition of these engineered particles to targeted region(s) of the airways (for example targeting to deep lung). It is more likely that the former will have similar deposition profiles, when inhaled and inspired, by patients whose inhalation flow rate is similar to that used to engineer the particle. This is in stark contrast to particles produced in the prior art where particle production is carried out independent (and without association) of the patient inhalation flow rate requirements and thus it is more likely that prior art particles will fail. Additionally, engineering particles using a mimicked respiratory airway and fluids (that is in line with the end user) within the mimicked respiratory airways confers stability of the particles to the environment that is in line with the end user. Another aspect of the second embodiment of the present invention is the application of the method of the invention which, for the same substance and the same inhalation flow rate it is possible to modify existing attributes or confer additional attributes to the engineered particles, in order to suit a specific patient inhalation flow rate, to suit a broad range of patient inhalation flow rates, improve deposition and targeting of the particles to the lower airways.

The third embodiment of the present invention is the application of the present invention whilst using the principle of effervescence to engineer and produce particles, to modify existing or to confer additional attributes to the engineered particles in order to suit a specific patient inhalation flow rate, to suit a broad range of patient inhalation flow rates and targeting the particles to the upper and lower airways. Another aspect of the third embodiment is the use of effervescence alone to engineer the particles. A yet further aspect of the third embodiment relates to the use of effervescent particles for inhalation therapy.

### First Embodiment

In order for the present invention to be understood the following guidelines for carrying out the method of the invention are given below, followed by more specific methods of utilising inhalation gas flow rate to engineer particles with attributes that make them more aerodynamically favourable for, at least, that inhalation gas flow rate.

To enable inhalation gas flow to efficiently engineer particles with attributes that make the particles aerodynamically favourable, it is preferable to adopt the following strategies:
a) De-aggregate and disperse the feedstock to form an aerosol whose aerosol particles are isolated from each other thus enabling the inhalation gas flow easy and ready access to all parts of the aerosol particles. The term aerosol as used in the context of the present invention is used to indicate a suspension of fine aerosol particles in a gas. The aerosol particle can be liquid droplets, frozen droplets, semi-wet particles, semi-dry particles, dry solid particles, and frozen solid particles and combinations thereof.
b) Use the inhalation gas flow rate to further de-aggregate and disperse the aerosol particles and carry the aerosol particles into preformed engineering environments and/or generate an engineering environment by vaporisation of liquids, into and through which the aerosol particles are carried by the inhalation gas flow. The engineering liquid that is used to form the engineering environment should, preferably, be present in small quantities but should also have high specific surface area. Examples of engineering environments with high specific surface area include, but are not limited to, gas(es), vapour(s), mist, steam, fog, spray and fine suspensions of liquid droplets in gas(es) or combinations thereof. The inhalation gas flow rate can trigger the release of such engineering environments and/or promote the conversion of liquid(s) into such engineering environments. The high specific surface area of the aerosol particles and the high specific surface area of the engineering environment coupled with the continuous and efficient mixing of the two, in laminar or turbulent inhalation gas flow facilitate the formation of uniform microenvironments containing a finite amount of the engineering environment around each aerosol particle, that is used to engineer that aerosol particle.
c) The inhalation gas flow rate forces the transfer of the engineering medium that is the contents of the associated microenvironment, onto, into and out of the associated aerosol particle at rates and to extents governed by the power of inhalation gas flow. The power of the inhalation gas flow is a combination of the intensity of the inhalation gas flow rate and the time over which this gas flow rate acts upon the aerosol particles. The power of the inhalation gas flow not only dictates the morphology and topography of the particles, it also ensures that these particles are also aerodynamically favourable for that inhalation flow rate. The action of the inhalation gas flow rate upon the high specific surface area of the aerosol particles facilitates rapid removal of the engineering medium out of the associated particles. The inhalation gas flow also rapidly removes any excess engineering medium thereby preventing bridging between the particles. Also, the resulting particles are almost dry upon impaction hence little or no drying is necessary. This is advantageous for heat-sensitive products. Additionally the engineering medium is only in contact with the aerosol particles for an extremely short time period thereby preventing bridging between aerosol particles whilst such short exposure times also minimises product degradation and thus this process is applicable to substance(s) which are sensitive to the engineering medium. The transfer of the engineering medium onto, into and out of the aerosol particle can proceed simultaneously or at different times depending on the feedstock and location of the engineering medium(s). The rate and extent of the transfer of the engineering medium onto, into and out of the aerosol particle is at least dependent upon the inhalation power, temperature, the specific surface area of the aerosol particles, the physico-chemical properties of the substance(s) of the aerosol particle and the engineering medium and the interaction between them. Additionally, the rate and extent of transfer of the engineering medium onto or into the aerosol particle is also dependent upon the specific surface area of the engineering medium. The greater the specific surface areas of the engineering medium the greater is the rate, magnitude and extent of this transfer. The removal of the engineering medium from the aerosol particles further facilitate “finger-printing” of the aerosol particle that further increases the specific surface area of the aerosol particles and thus this increase in the specific surface area can further and perpetually facilitate the transfer of the engineering medium onto, into and out of the aerosol particle. “Finger prints” can be classified as microscopic changes, one of which is morphological (shape, form and dimensions) and the other is topographical (such as depressions, dents, indentations, ridges, channels, formation entrances and exits into and out of the particle and breakages of the particle). These are examples of “fingerprints” given to exemplify the impact of the power of the inhalation gas flow and should not be seen as the only “fingerprints” that can be generated by the inhalation gas flow rate. “Finger prints” can be classified as microscopic changes, one of which is morphological (shape, form and dimensions) and the other is topographical (such as depressions, dents, indentations, ridges, channels, formation of entrances and exits into and out of the particle and breakages of the particle).
d) Dispersed, individual aerosol particles can collect a substantially greater quantity of engineering medium at a faster rate, at any one time, compared to un-dispersed particles, thereby, substantially reducing the exposure time between the aerosol particles and the engineering medium whilst still ensuring the absence of aerosol particle clumping, fusion and agglomeration. If the engineering medium has high specific surface area, a small amount of engineering medium can be used to engineer a large amount of aerosol particles, in an efficient manner, compared to conventional practises such as crystallisation from solution. This also has additional benefits of reducing the wastage of the engineering medium. Additionally, reducing exposure time of the aerosol particles to the engineering medium, has several advantages in that any engineering medium can be used whether it is a solvent or non-solvent for the substance(s) of the aerosol particle in order to obtain partial or complete aerosol particle solubility or insolubility. Short exposure time also minimises, product degradation and thus the process of the invention is applicable to substance(s) which are sensitive to the engineering medium.
e) Additionally, inhalation gas flow draws in all the aerosol and thus ensures maximum yield and maximum deposition to the target regions.
   These strategies enable the particles to be engineered in an uniform manner and will consequently give a final product that is uniform, monodisperse and of defined shape, forms and dimensions. The addition of heat is not required thus making the process suitable for heat-sensitive materials. Furthermore engineering may continue after particle impaction to allow the formation of particles of larger in size compared to the starting aerosol particles whilst maintaining the monodispersity of the particles, which are non-cohesive and thus this is important for engineering particles larger than the starting aerosol particles. The inhalation gas flow can continue after aerosol particle impaction and can be used to stir a liquid bed engineering medium. This stirring is non-aggressive, non-destructive and facilitates particle suspension and separation in this liquid bed whilst still enabling the formation of crystalline and monodisperse particles of desired size. Crystalline, monodisperse particles can be obtained up to any size depending upon the stirring imparted to liquid bed and thus to the particles by the inhalation gas flow rate as well as the time within the engineering medium, the type of liquid bed, temperature and exposure time of the particles to the liquid bed.

The system for using inhalation gas flow rates to engineer the particles of the present invention basically consists of three parts:
a) A device to deliver the feedstock.
b) Inhalation flow zone that can consist, at least, a respiratory system mimicking the airways (at least in terms of the geometry and fluids contained therein) and/or a spacer device.
c) System that generates the inhalation gas flow rate.

The device to deliver the feedstock can also be part of the inhalation flow zone. FIG. 1 is a general representation of the process in accordance with the present invention. The inhaled gas flow (10) through the inhalation flow zone (40) is established and adjusted to the desired flow rate, using the vacuum system (60). The delivery device (20) then provides the feedstock (30) in bulk or as an aerosol to the inhalation gas flow. The inhalation gas flow acts to aerosolise bulk feedstock (from breath activated devices) and further de-agglomerate and disperse the aerosol and carry the aerosol into and through the inhalation flow zone, where any engineering medium(s) contained within the inhalation flow zone can be used by the inhalation power to engineer and produce particles with attributes matched with that inhalation gas flow rate used to engineer them whilst simultaneously or subsequently testing the resulting particles to ensure high deposition in one or more specific target regions, in-vitro, in the mimicked respiratory system.

The inhaled gas (10), that forms part of the inhalation gas flow, used to carry the aerosol into and through the inhalation flow zone can be, but is not limited to, air, the individual gas components of air such as but not limited to the gases of Nitrogen, Oxygen, Argon, Helium, carbon dioxide and the like and their combinations. A suitable gas is air.

In a preferred aspect of the first embodiment, the environment containing the engineering medium can be preformed before being inhaled into the inhalation flow zone. The preformed gas can contain an environment that is loaded with the vapour of one or more liquids before entering into the inhalation flow zone. The vapour of in the gas can have any percentage (%) relative humidity whether dry i.e. % relative humidity less than 10% or moist i.e. % relative humidity equal or greater than 10%. In a preferred aspect of the first embodiment, the inhaled gas can contain an environment loaded with the vapour of water before entry into the inhalation flow zone. In another aspect, the inhaled gas is dry in terms of its water vapour content.

In a preferred aspect of the present invention, the temperature of the inhaled gas can aid the inhalation flow rate to engineer the aerosol particles by improving the surface area of the engineering medium of the microenvironment thereby improving the rate and extent of transfer of the engineering medium onto, into and out of the aerosol particles. Further preferably, the temperature of the inhaled gas can be ambient or cool i.e. below +25° C. or hot i.e. at or above 25° C.

In another preferred aspect of the first embodiment, the pressure of the gas before it is inhaled can be altered i.e. increased or decreased above or below 1 bar to facilitate particle engineering as alteration of the pressure of the gas can improve the specific surface area of the aerosol particles and the engineering medium.

The inhalation gas flow rate can range from 1 to 1000 L/min, preferably from 15 to 500 L/min and more preferably from 15 to 200 L/min and most preferably from 15 to 120 L/min. In a preferred aspect of the invention, the inhaled gas can be cooled or heated in order to facilitate the engineering of the aerosol particles.

It should be appreciated that the inhalation flow rate used to engineer the particles is not the same as the inhalation flow rate used to test the particles. Suitable devices which can be used to increase or decrease the inhalation gas flow to comply with the inhalation flow rate requirements for testing can be used. An example of such device can be a mixing inlet valve.

The feedstock to be engineered by the present invention can be in the solid state, the liquid state, the vapour state and combinations thereof. The feedstock can be static or in motion, dispersed or in bulk before delivery to the inhalation flow zone.

Where the feedstock is in the solid state it can be produced by various methods including but not limited to: spray drying, micronisation, granulation, sieving, fractioning, freezing, freeze drying, spray freezing, spray freeze drying, spray-chilling, spray congealing, spray cooling, freeze fracturing, spray freeze fracturing, emulsion solvent evaporation/extraction, coacervation, extrusion spheronisation, coating of nonpareil spheres, pelletization, wet granulation, dry granulation, crystallisation. The starting solid feedstock may be dry or wet. The shape, source and phyico-chemical properties of the starting feedstock is unimportant as long as the solid feedstock is capable of being aerosolised. Suitable solid feedstock are spray-dried, micronised, granulated, sieved, fractioned, spray freeze-fractured and crystallised products.

Where the starting feedstock is in the liquid state it can comprise one or more liquids, the liquid feedstock may have one or more substances dissolved, suspended in the liquid(s) and combinations thereof. The liquid of the feedstock may comprise volatile or non-volatile liquids and combinations thereof. Where there are more than one liquid in the liquid feedstock, the liquids may be immiscible, miscible or only partially miscible. More specific examples of suitable liquids include but are not limited to: water; alkaline solutions or suspensions of potassium bicarbonate, calcium carbonate, sodium bicarbonate, calcium carbonate, sodium glycine carbonate, ammonium carbonate, hydrocarbons solvents; mineral spirit; mineral oils; halogenated solvents, such as methylene chloride and bromide, freons, bromo-chloro-methane, chloroform and carbontetrachloride; oxygenated solvents, such as ketones, ethers, esters, carboxylic acids, aldehydes, alcohols and carbonates; nitrogen containing solvents, such as amines and amides; sulphur containing hydrocarbon solvents, such as sulphoxides and sulfonates; and other hetero-atoms containing hydrocarbon solvents; acidic solutions of citric acid, tartaric acid, acid citrate, acid phosphates, ascorbic acid, acid drugs, mineral acids, such as hydrochloric acid, hypochlorous acids, sulfonic acids, sulphuric acids, phosphoric acids, nitric acids and anaesthetics such as halothane, enflurane, isoflurane, methoxyflurane, sevoflurane,. Yet more specific examples are liquefied gases e.g. liquid nitrogen (boiling point −196° C.), liquid oxygen (boiling point −183° C.), liquid argon (boiling point −186° C.), chlorofluorocarbons, fluorocarbonated refrigerants (such as dichlorodifluoromethane , perfluoropropane , CF4, C2F6, C3F8, C4F8, C2F4, C3F6), hydrofluoroalkanes (such as HFA-134a, HFA-227) or any liquid and combinations thereof. A suitable acid is citric acid and a suitable alkali is sodium bicarbonate.

Preferably a suitable liquid is water, a suitable ketone is acetone; a suitable alcohol is ethanol, a suitable hydrofluoroalkane is HFA-134a and a suitable liquefied gas is liquid nitrogen.

The liquid feedstock can be introduced in an atomised form such as but not limited to mist, droplets, foam, spray, steam, fog or vapour.

Preferably the substance or substances to be engineered by the process of the present invention can include but is not limited to therapeutic substances, prophylactic substances, diagnostic substances or an excipient. It is also appreciated that more than one of such substances may be used in combination to create the engineered particles of the present invention. Other materials commonly used in pharmaceutical compositions, such as diluents, flavourants, fragrances, dyes, nutrients and sweeteners are also considered as possible substances within the understanding of the present invention.

Suitable nutrients include: retinoids such as all-cis retinoic acid, 13-trans retinoic acid and other vitamin A and beta carotene derivatives, vitamins D,E,K and water insoluble precursors and derivatives thereof.

The therapeutic substances, prophylactic substances and diagnostic substances of the present invention are preferably taken from the group comprising: peptides, proteins, organic substances, inorganic substances, pro-drugs, antigens and hormones.

More specific examples of substances that can be treated under the present invention include: corticosteroids; anti-inflammatories such as Beclomethasone, betamethasone, Fluticasone, flunisolide, budesonide, dexamethasone, tipredane, triamcinolone acetonide; anti-tussives such as noscarpine; and bronchodilators such as ephedrine, adrenaline, fenoterol, formoterol, isoprenaline, metaproterenol, phenylephrine, phenyl propanolamine, pirbuterol, reproterol, rimiterol, salbutamol, salmeterol, formoterol, terbutaline, isoetharine, tulobuterol, orciprenaline and (−)-4-amino-3,5-dichloro-α[[[6-[2-(2-pyridinyl)ethoxy}hexyl]amino]methyl]benzenemethanol,

Further specific examples of suitable substances include: the diuretic amiloride; anticholinergics such as ipratropium, ipatropium bromide, atropine, oxitropium and oxitropium bromide; hormones such as cortisone, hydrocortisone and prednisolone; and xanthines such as aminophylline, choline theophyllinate, lysine theophyllinate and theophylline.

Yet further specific examples of suitable substances include: analgesics such as codeine, dihydromorphine, ergotamine, fentanyl and morphine; diltiazem which is an anginal preparation; antiallergics such as cromoglycate, ketotifen and nedocromil; anti-infectives such as cephalosporin, penicillins, streptomycin, sulphonamides, tetracyclines and pentamidines; and the anti-histamine methapyrilene.

Yet further specific examples still include: anti-neoplastic substances like bleomycin, carboplatin, methotrexate and adriamycin; amphotericin B; anti-tuberculous substances such as isoniazide and ethanbutol. Therapeutic proteins and peptides(e.g. insulin and glucagon, prostaglandins and leukotrienes) and their activators and inhibitors including prostacyclin (epoprostanol), and prostaglandins E, and E2 are also considered to make suitable substances for treatment using the method of the present invention.

It will be appreciated to the artisan that, where appropriate, the above listed therapeutic substances may be used in the form of salts (e.g. as alkali metal or amine salts or as acid addition salts) or as esters (e.g. lower alkyl esters) or as solvates (e.g. hydrates) to optimise the activity and/or stability of the therapeutic substance.

Preferably, where the substance is a therapeutic substance it will either be an anti-inflammatory drug or a bronchodilator. More specifically the preferred therapeutic substances of the present invention are beclomethasone dipropionate, Fluticasone propionate, budesonide, salmeterol xinafoate and salbutamol sulphate. More specifically, where the substance is a protein or peptide, the preferred protein or peptide is bovine serum albumin or insulin.

Preferably, when the excipient is used on its own to produce particles and not in combination with any other type of substance (i.e. therapeutic agents, prophylactic agents and diagnostic agents) such excipients are sugars, preferably taken from the group comprising: monosaccharide, disaccharide, polysaccharide and sugar alcohols such as sorbitol, mannitol, maltitol. Further preferably the excipient is lactose.

It is also appreciated that more than one of the above substances may be used in combination to produce the particles of the present invention. Suitable combinations comprise a short acting β₂ agonist and an antimuscarinic, typically salbutamol and ipatropium bromide; or fenoterol and ipatropium bromide. Alternatively the combination of a short acting β₂ agonist and a corticosteroid in the form of salbutamol and beclomethasone, salbutamol and Fluticasone are advantageous. A further alternative is the combination of a long acting β₂ agonist and a corticosteroid, typically salmeterol and Fluticasone; or formoterol and budesonide.

As discussed above, the combination of one or more therapeutic, prophylactic or diagnostic substance (as listed above) with one or more pharmaceutical excipients is also considered desirable within the present invention. The excipients suitable to be used in combination with therapeutic substance are not necessarily the same as those that are appropriate when a particle is produced from an excipient alone.

The presence of an excipient in combination with therapeutic substances can facilitate a retarded, controlled, sustained or targeted release of the therapeutic, prophylactic or diagnostic substance. According to the present invention, suitable excipients to regulate the release, are preferably either non biodegradable, biodegradable or bioerodible polymers.

More specifically, suitable polymers include but not limited to: cyclodextrins and derivatives thereof, sodium caseinate, dipalmitoyl phosphatidyl choline (DPPC), human serum albumin, phospholipids, cellulose acetate phthalate, hydroxypropyl methylcellulose phthalate, ethyl cellulose, hydroxypropyl methyl cellulose, hydroxyethyl cellulose, hydroxypropyl cellulose, ethyl hydroxyethyl cellulose, carboxymethyl cellulose, methyl cellulose, cellulose acetate butyrate, poloxamer, poly(lactic acid), poly(lactic-co-glycolic acid), poly(lactide)s, poly(glycolide)s, poly(lactide-coglycolide)s, poly(p-dioxanones), poly(caprolactone), polycarbonates, polyamides, polyanhydrides, poly(alkylene alkylate)s, polyamino acids, polyhydroxyalkanoates, polypropylenefumarates, polyorthoesters, polyacetals, polyacrylamides, polycyanoacrylates, polyalkylcyanoacrylates, polymetha polyphosphate esters, polyphosphazene, polyurethanes, polyacrylates, polymethacrylate, poly(methyl methacrylate), poly(hydroxy ethyl methacrylate-co methyl methacrylate), carbopol 934, ethylene-vinyl acetate and other acyl substituted cellulose acetates and derivatives thereof, polystyrenes, polyvinyl alcohol, polyvinyl pyrrolidone, polyvinyl chloride, polyvinyl fluoride, poly(vinylimidazole), chlorosulphonated polyolefins, polyethylene, polyethylene glycols, polypropylene, polyethylene oxide, copolymers and blends thereof.

Preferably, the selected polymer is biocompatible in that it degrades or erode in-vivo to form non-toxic small molecules. More preferably, the biocompatible polymer is pharmaceutically acceptable for delivery to the respiratory tract. Even more preferably, the polymer is both pharmaceutically acceptable to the lung and has therapeutic properties.

The substance or substances to be engineered might contain one or more stabilisers to protect the therapeutic substances from degradation and maintain the biological activity. The term stabilisers as described herein means any substance which binds or interacts in a covalent or a non-covalent manner with the therapeutic, prophylactic, diagnostic substance or excipient. Suitable stabilisers that can be used in the present invention will be appreciated by the skilled man (see for instance U.S. Pat. No. 5,716,644; 5,674534; 5,654,010, 5,711,968; 6,284,283). However, preferred stabilising substances include: sucrose, trehalose, polyvinyl pyrrolidone and dextran.

It is appreciated that the substance in the particles and/or in the liquid can have preservative, antiseptic, disinfection and/or sterilisation properties. Suitable preservative, antiseptics, disinfectants and sterilising substances include but are not limited to: phenolics (such as: phenol, cresols, xylenols), halogenated phenolics (such as, chlorocresol, chloroxylenol, hexachlorophene, triclosan), alcohols (such as, ethanol, benzyl alcohol, bronopol, phenoxy-ethanol), aldehydes (such as, formaldehyde, glutaraldehyde), organic acids and their ester, quaternary ammonium compounds (such as, cetrimide, benzalkonium chloride), biguanides (such as chlorhexidine, polyhexamethylene biguanide), amidines (such as, propamidine, dibromopropamidine), halogens and their compounds (such as, hypochlorous acid, Eusol, Chloronated Soda solution, chloramines T, halozone, potassium iodide, iodophores, betadine), Metal ions (such as, mercury, silver, aluminium, phenylmercuric nitrate and acetate, thiomersal), acridines (such as, aminacrine hydrochloride), gases (such as ethylene oxide, formaldehyde, β-propiolactone, propylene oxide, methyl bromide, gas plasmas [under vacuum or at atmospheric pressure] in combination with heat or radiation).

The delivery device (20); can be a device containing bulk powder, frozen particles, bulk liquid or particles suspended in a bulk liquid. Such devices containing bulk feedstock can be breath actuated in that it uses the flow of gas, resulting from the vacuum system, to aerosolise the feedstock from the delivery device. The delivery device can be capable of generating an aerosol of finely divided liquid droplets, solid particles, mist, vapour, fog, steam and the like and combinations thereof. The delivery device may be self actuated delivery devices using the energy supplied by the device for example from compressed gases (Nitrogen, Oxygen, Argon, Helium, Carbon dioxide, Air and the like), liquefied propellants (such as dichlorodifluoromethane, perfluoropropane, CF4, C2F6, C3F8, C4F8, C2F4, C3F6), hydrofluoroalkanes (such as HFA-134a, HFA-227) or any liquid mediums that can generate a propulsive vapour, propulsive gas and combinations thereof. The delivery device can be a metered or non-metered delivery system providing the feedstock continuously or discontinuously. Delivery devices for liquid feedstock include, but is not limited to Metered Dose Inhalers (MDI's), nebulisers or any other devices for atomising liquid feed stock into droplets using pneumatic, ultrasonic or pressure nozzles, rotary atomisers, spinning disks, blow cans, gauge needles, electro spray devices, spray guns or airbrushes. Powder delivery devices can include but is not limited to Dry powder inhalers (DPI's) powder spray devices, powder guns, dispersion nozzles or insufflators. The delivery device can be any equipment that can generate the foregoing.

Suitable delivery devices for aerosolising liquid feedstock are typically an airbrush, a nebuliser and metered dose inhalers. Suitable atomisation gases for the airbrush or nebuliser include, but is not limited to, air, the individual gas components of air such as but not limited to the gases of Nitrogen, Oxygen, Argon, Helium, carbon dioxide and the like and their combinations. A suitable atomisation gas is air. The atomisation gases need not necessarily be the same as the gas(es) comprising the inhalation gas flow (10). It is known that the pressure of the atomising gas and the flow rate of liquid feedstock to the atomising chamber controls the droplet size of the aerosol particles. In a preferred aspect, the pressure of the atomising gas used to atomise the liquid feed stock is at a pressure range of 0.5 bar to 15 bars, preferably between 1 bar to 8 bars, more preferably between 1 bar to 4 bars and most preferably between 1 bar to 2.5 bars. In a preferred aspect, the flow rate of the liquid feedstock to the atomisation chamber is from 0.5 ml/minute to 100 ml/minute, preferably from 0.5 ml/minute to 20 ml/minute and most preferably for 0.5 ml/minute to 5 ml/minute. Suitable delivery devices from which the solid feedstock is aerosolised are dry powder inhalers or the inhaler glass device which is a 29 Quick-fit socket to fit with the mouth of the induction port of twin stage impinger, cascade impactor, oral cast and the like, or any induction port that can simulate the mouth and throat of any mammalian.

One or more feedstock can be introduced by one or more delivery devices. The devices can be the same or different devices. Where an atomisation gas is used the atomisation protocols such as the gas constituents and atomisation pressure need not be the same from one delivery device to the other. These devices can introduce the feedstock at any angle(s) to the direction of the inhalation gas flow. Preferably one feedstock is introduced to the inhalation gas flow. Preferably the angle of introduction are 0°, 90°, 180° and 270° e.g. tangential, co-current, counter-current or cross-current to the direction of the inhaled gas flow and more preferably co-current to the direction of the inhaled gas flow.

The inhalation flow zone (40) lies between the delivery device and the vacuum system and can consist of a plurality of different parts, which need not necessarily be physically connected.

One part of the inhalation flow zone can be a spacer device that channels the aerosol, reduce the velocity of the self-actuated aerosol so that the inhalation flow rate can control the velocity of the aerosol, maximise aerosol delivery to the next part of the inhalation flow zone, removes some oversize and agglomerated aerosol particles, de-agglomerate the aerosol particles, increase the specific surface area of the aerosol particles, facilitate removal of the engineering medium from the aerosol particles and “fingerprint” the aerosol particles with attributes. These attributes can further maximise the aerosol delivery to the next part of the inhalation flow zone, facilitate further dispersion of the aerosol particles, further increase the specific surface area of the aerosol particles, accelerate and further facilitate aerosol particle engineering in the same part or other parts of the inhalation flow zone.

The spacer device can be of any shape and dimensions (tube, cone shaped and the like) and made of any material (plastic, glass, metal, which could be conductive and non-conductive with regards to heat, electricity and radiation). The spacer device may have additional ports that can facilitate introduction or removal of one or more feedstock. These ports can also be used to introduce one or more gases, vapours, liquefied gases, or other aerosols (that are in vapour, liquid, solid or frozen states). Hence any substance can be introduced via these ports that can facilitate the particle engineering process and add to or modify the existing aerosol particle attributes which the inhalation flow rate on it's own is incapable of generating in order to enhance or modify the delivery capabilities of the resulting particles.

Suitable spacer devices can be a volumatic®, copper and glass tubes with internal diameters from 1 mm to 1000 mm and preferably from 20 mm to 28 mm and length above 10 metres, preferably with lengths below 10 m, more preferably with lengths from 1 cm to 10 m and most preferably from 10 cm to 100 cm and can be made from copper or glass. The length of the spacer device can be selected to enable the inhalation gas flow rate sufficient time to engineer the aerosol particles. The spacer device can have one or more inlet and one or more outlet ports. In a preferred aspect the spacer has 2 inlet ports and 1 outlet port, in another preferred aspect, the spacer has one inlet port and one outlet port. In a preferred aspect, one of the inlet ports can be connected to chamber(s) that contains a liquid or combinations of liquids.

More specific examples of suitable liquids, in this chamber or within the spacer device, include but are not limited to: water; alkaline solutions or suspensions of potassium bicarbonate, calcium carbonate, sodium bicarbonate, calcium carbonate, sodium glycine carbonate, ammonium carbonate, hydrocarbons solvents; mineral spirit; mineral oils; halogenated solvents, such as methylene chloride and bromide, freons, bromo-chloro-methane, chloroform and carbontetrachloride; oxygenated solvents, such as ketones, ethers, esters, carboxylic acids, aldehydes, alcohols and carbonates; nitrogen containing solvents, such as amines and amides; sulphur containing hydrocarbon solvents, such as sulphoxides and sulfonates; and other hetero-atoms containing hydrocarbon solvents; acidic solutions of citric acid, tartaric acid, acid citrate, acid phosphates, ascorbic acid, acid drugs, mineral acids, such as sulfonic acids, sulphuric acids, phosphoric acids, nitric acids and anaesthetics such as halothane, enflurane, isoflurane, methoxyflurane, sevoflurane. Yet more specific examples are liquefied gases e.g. liquid nitrogen (boiling point −196° C.), liquid oxygen (boiling point −183° C.), liquid argon (boiling point −186° C.), chlorofluorocarbons, fluorocarbonated refrigerants (such as dichlorodifluoromethane, perfluoropropane , CF4, C2F6, C3F8, C4F8, C2F4, C3F6), hydrofluoroalkanes (such as HFA-134a, HFA-227) or any liquid and combinations thereof. The liquid in the chamber or within the spacer device can have one or more substances dissolved or suspended within the liquid. The substance can be any of the foregoing and may be the same or may be different from the substances of the feedstock.

Preferably a suitable liquid is water, a suitable ketone is acetone, a suitable alcohol is ethanol and a suitable liquefied gas is liquid nitrogen, and a suitable acid is citric acid and a suitable alkali is sodium bicarbonate.

In a preferred aspect where a liquid, in a chamber, connected to a spacer, the inhalation gas flow serves to aspirate the liquid and generate an environment (i.e. vapour, gas, or suspended liquid droplets) within the spacer through which the inhalation gas flow carries the aerosol particles into so as to initiate aerosol particle engineering before the main aerosol particle engineering in the mimicked airway.

In a preferred aspect of the present invention, the temperature in one or more parts of the spacer device can be controlled and/or changed and/or maintained in the temperature range −200° C. to +200° C. It should be clear that the temperature in one part of the spacer device may be the same or different from any other parts of the spacer device. A more preferred temperature range for any part of the spacer device is −50° C. to +120° C., a further preferred temperature range is between −20° C. to +50° C., a yet more further preferred temperature range is +20° C. to +50° C. and most preferably at mammalian body temperature in the range +34° C. to +42° C. In a preferred aspect the temperature in different parts of the spacer device can be controlled and/or maintained by, but not limited to, conductive, convection and radiant heating.

Another part of the inhalation flow zone, is a simulated human airway geometry (or mimicked airway), connected via an induction port (this mimics the human mouth and throat) either to the delivery device or spacer device. The mimicked airway can be composed of interconnected sections, each section representing deeper stages of the in-vivo respiratory system. The mimicked airway can be real or artificial. Suitable, artificial, simulated human airway geometries include but are not limited to inertial impactors. The impactor is designed such that as the aerosol stream passes through each stage, particles having a large enough inertia will impact upon that particular stage plate, whilst smaller particles will pass to the next impaction stage. Suitable impactors include but are not limited to that of an Andersen cascade impactor, twin-stage impinger, multistage liquid impinger, electronic lung, hydraulic lung, artificial airways or any device that can mimic or simulate the respiratory system of mammalians and can be used in part or in whole depending on the intended target region and/or particle attributes required can be used as a mimicked airway. The mimicked airway can be of any geometry, any shape or form, of any dimensions, it can contain one or more compartments and be a single stage or multi-stage device. The mimicked airway can operate at constant or variable inhalation gas flow rates.

A preferable mimicked airways is that provided by an Anderson cascade impactor and twin stage impinger. The stage plates of the Andersen cascade impactor simulate the pharynx, trachea & primary bronchi, secondary bronchi, terminal bronchi and alveoli.

The mimicked airway is a device that is normally only used for measuring the fine particle dose and size of the aerosol cloud. Whereas, this mimicked airway is used, primarily, in this invention as an engineering device and secondly as testing device to ensure that engineered particles deposit in the region(s) of the mimicked airways according to the inhalation gas flow rate and the morphologies, topographies and aerodynamic diameter dictated to the particles by the inhalation gas flow rate.

In choosing the correct set of interconnected sections it is possible to engineer the particles such that they deposit highly and target the naso-pharynx, oropharynx, any part of the conducting zones of the airways, including the trachea, bronchi, bronchioles and terminal bronchioles or any locations within the respiratory zones of the airways including bronchioles, alveolar ducts or alveolar sacs and combinations thereof.

The mimicked airway can contain one or more fluid(s) to reflect the environment within the in-vivo respiratory system. The fluid(s) within the inhalation flow zone can be physiological body fluids (can be but is not limited to, for example water, saline, mucus, any liquids present in any mammalian body cavities) or non-physiological body fluids (any fluid which facilitates particle engineering). These fluids can exist is gaseous phase, vapour phase, liquid phase and combinations thereof. The gaseous phase, vapour phase and liquid may or may not be of the same composition. The fluid may be solvent (i.e. the aerosol particles are partially or completely soluble) or anti-solvents (i.e. the aerosol particles are partially or completely insoluble) for the substance(s) of the aerosol particles. This fluid can act as a body fluid to mimic the environment that is more in line with the conditions in the patient airway of the end user and/or an engineering medium for the aerosol. The fluid(s) can be in separate stages of the inhalation flow zone or can co-exist in the same environment. Where the fluid is present in more than one part of the inhalation flow zone the fluid(s) within each part can be the same or differ from each other.

More specific examples of suitable fluid(s), in the mimicked airway include but are not limited to: water; saline, mucus, alkaline solutions or suspensions of potassium bicarbonate, calcium carbonate, sodium bicarbonate, calcium carbonate, sodium glycine carbonate, ammonium carbonate, any liquids or liquids mimicking those present in any mammalian body cavities), hydrocarbons solvents; mineral spirit; mineral oils; halogenated solvents, such as methylene chloride and bromide, freons, bromo-chloro-methane, chloroform and carbontetrachloride; oxygenated solvents, such as ketones, ethers, esters, carboxylic acids, aldehydes, alcohols and carbonates; nitrogen containing solvents, such as amines and amides; sulphur containing hydrocarbon solvents, such as sulphoxides and sulfonates; and other hetero-atoms containing hydrocarbon solvents; acidic solutions of citric acid, tartaric acid, acid citrate, acid phosphates, ascorbic acid, acid drugs, acids such as hydrochloric acid, hypochlorous acid and mineral acids such as sulfonic acids, sulphuric acids, phosphoric acids, nitric acids and anaesthetics such as halothane, enflurane, isoflurane, methoxyflurane, sevoflurane,. Yet more specific examples are liquefied gases e.g. liquid nitrogen (boiling point −196° C.), liquid oxygen (boiling point −183° C.), liquid argon (boiling point −186° C.), chlorofluorocarbons, fluorocarbonated refrigerants (such as dichlorodifluoromethane, perfluoropropane, CF4, C2F6, C3F8, C4F8, C2F4, C3F6), hydrofluoroalkanes (such as HFA-134a, HFA-227) or any liquid and combinations thereof.

Preferably a suitable fluid(s) is water, a suitable ketone is acetone, a suitable alcohol is ethanol and a suitable liquefied gas is liquid nitrogen.

The temperature of mammalian respiratory system ranges from about +34° C. to about +42° C. Hence the process of the present invention enables engineering of the particles, preferably, within this temperature range, however, temperatures outside of this range can be used.

Movement of air into and out of the lungs is driven by pressure differentials or gradients across the lungs. When respiratory muscles (diaphragm and intercostals muscles) contract to expand the thoracic cavity, a force is applied to the lung surface which causes expansion of the lungs. Lung expansion occurs because the lungs are compliant and distensible. By expanding, a negative pressure is created within the lungs, specifically in airways and alveoli. Air flows down it's pressure gradient into the airways and alveoli, which now have a lower pressure relative to the external atmospheric pressure” (Altiere, R. J. and Thompson, D. C., Pulmonary physiology and Pharmacology, chapter 4, page 85, Inhalation Aerosols, Physical and Biological Basis for Therapy, Hickey, A. J., Marcel Dekker, Inc, 1996). The mammalian respiratory system can act as a vacuum pump during inhalation. In this invention the pressure gradient that generates and causes air to flow into the inhalation flow zone and thus the mimicked airway was supplied by a vacuum pump. Other examples include turbines, fan assisted and aspirators, vacuum cleaners, cyclones, suction devices, air pumps, and the like. It is clear that any device that can generate a constant or variable inspiration flow rates as demanded by the engineering requirements and testing conditions can be used without departing from the scope of the invention.

The gas that flows into the inhalation flow zone can be inhaled, inspired, insufflated, aspirated, drawn into, withdrawn, breathed in, pulled, taken in, can be generated by pressure gradients, differential pressure, negative pressure, the result of a wind or of a atmospheric depression, vacuumed, pumped out.

In a preferred aspect of the first embodiment, the inhalation gas flow is used to engineer Salbutamol sulphate particles from a non-volatile liquid that is water at inhaled gas temperatures below +50° C. Since water has a boiling point of +100° C., the process of the present invention can engineer particles from liquid feedstock at temperatures below the boiling point of the liquid in the liquid feedstock. Since pharmaceutical substances tend to be heat-sensitive, this process is thus extremely suitable for such materials. Equally the process of the invention can therefore be used to engineer particles at temperatures equal or above the boiling point of the liquid of the feedstock.

In a preferred aspect of the first embodiment, the aerosol particles are engineered while they are suspended in a gas or vapour. In another preferred aspect the inhalation gas flow deposit the isolated aerosol particles in a liquid bed.

In another aspect, one or more liquid mediums can be introduced in any stage of the mimicked respiratory system to collect deposited aerosol particles. The turbulence within the liquid media resulting from the action of the inhalation gas flow rate upon these liquid media serves to agitate, stir and separate deposited particles thereby preventing particle clumping whilst promoting crystallisation and particle growth of the deposited particles. By adjusting the inhalation gas flow rate, it is possible to adjust the turbulence within the liquid media and thus adjust the agitation and stirring imparted to the liquid media. This enables control of the particle dispersion, the size of the particles and the rate of particle growth whilst maintaining a stirring rate compatible with delicate particles that are unsuitable for mechanical stirring. Furthermore, the particles are dispersed, monosized and consequently have the same settling velocity. The settling velocity of the particles in the engineering liquid is reduced as a result of stirring caused by the turbulent air flow, hence, this maintains full contact with the liquid, preventing contact with the sides of the glassware of the device hence the particles will grow in a uniform manner whilst maintaining its regular shape and monodispersity compared to conventional crystallisation, where fracture caused by the stirring blades and adherence of the particles to the blade causes variations within the batch and from batch to batch. Hence this is a method of collected non-useful particles by growing them to increase their size to one which is more suitable for the application and hence increasing the overall particle yield. Particles engineered in this way are larger in size than the starting deposited particles and their shape and monodispersity is maintained. The particle size and particle growth rate, within the liquid medium can be promoted by heating the liquid medium. Additionally the surface texture of the particles can be engineered to suit the intended use. At the same time the engineered particles can be more crystalline compared to the particles before deposition. The liquid medium used to collect the deposited particles may or may not have substances, as described in the foregoing, dissolved or suspended within it

In a preferred aspect of the present invention, the inhalation power dictates particle attributes. In another aspect of the present invention the inhalation gas flow rate dictates particle attributes. These attributes can be but are not limited to morphology, topography and aerodynamic diameter to give particles that are more favourable for at least that inhalation flow rate. Suitable morphological attributes can be but are not limited to particle size, shape, volume and surface area. Suitable shapes include but are not limited to cubes, cuboidal, spheres, hemi-spheres and fibre-like shapes and combinations thereof. Suitable particle size can range from at least 0.5 μm to 80 μm. Suitable topographies include but are not limited to controlling surface regularity either by increasing surface smoothness, increasing surface roughness such as but not limited to the formation of “hairs”, causing depressions, golf ball like particles, dents, indentations, cat's tongue, ridges, sharkskin, striae, channels, entrances and exit into and out of the particles. The “aerodynamic diameter” as used in this present invention means the diameter of a sphere of unit density that has the same terminal sedimentation velocity in air under normal atmospheric conditions as the particle in question.

In another aspect of the present invention one section of the mimicked respiratory system enables engineering and targeting of particles with aerodynamic diameter from 0.4 μm to 0.7 μm. This enables the aerodynamic diameter to be controlled to an accuracy of at least 0.3 μm.

In a preferred aspect of the present invention, the topography of the particles can be controlled without affecting the morphology of the particles. In a preferred aspect of the present invention, the morphology of the particles can be controlled without affecting the topography of the particles. In a further preferred aspect of the present invention, the morphology and topography of the particles can both be controlled.

The morphological and topographical attributes of the engineered particles can give these particles high specific surface area and this is suitable for hydrophobic drugs whose dissolution is rate-limiting step for their absorption.

In another aspect of the first embodiment, increases in the specific surface area of the aerosol and engineering medium are preferable to enable fast and efficient particle engineering. The increase in specific surface area of the aerosol particles may result from increase in the aerosol particle velocity, particle dispersion or de-aggregation before the inhalation flow zone or within the inhalation flow zone, or the power of the inhalation gas flow. The aerosol velocity can be zero as form a breath-actuated device or at low velocity as from example a nebuliser or at high velocity as from a atomiser. An increase in aerosol particle velocity improves aerosol particle dispersion and thus increased aerosol specific surface area. An increase in specific surface area can be facilitated by a long travel time in the inhalation flow zone as a result a spacer device that is sufficient long can be used to allow aerosol de-aggregation and formation of microenvironments around each individual aerosol particles rather than around agglomerated aerosol particles so as to enable the inhalation flow rate to act and engineer single individual particles rather than a group of agglomerated particles which consequently have lower specific surface area. An increased specific surface area of the aerosol and engineering medium facilitate fast and easy addition to and removal from the aerosol particles of the engineering medium. It also facilitates fast and easy removal of bound and unbound liquids from the aerosol particles wherein unbound liquids are liquids that can be readily removed whilst bound liquid are liquids that can not be readily removed.

The process of the current invention successfully produced particles form bovine serum albumin, hence it can be applied to engineer other macromolecules.

It is clear to the skilled artisan that the particles produced by the invention can be used on their own or as part of formulations. Although the particles are primarily intended for pharmaceutical formulation purposes, it can be used for purposes other than pharmaceutical. When particles are used for inhalation, the particles of the invention can be filled into bulk storage chambers such as multidose reservoirs or unit dose containers such as cartridges or blister packs. Other suitable routes of administration include but are not limited to oral, parenteral, nasal, rectal, tonsillar, buccal, intra-ocular, topical/transdermal or vaginal.

### Second Embodiment

The second embodiment of the present invention more specifically utilises patient inhalation flow rate coupled with a mimicked respiratory airway to enable not only engineering of the particles but also testing of the particles under the conditions that the particles would normally be tested. This assures the formation of particles with aerodynamic diameters that are more favourable to the patient inhalation flow rate whilst enabling maximum deposition of the resulting engineered particles to the targeted regions of the airway. Additionally, using body fluid(s) present in the lungs, as an engineering medium enables the engineered particles to be stable to the engineering environment that is the body fluid(s). Hence these particles are more stable once inhaled by the end user (the patient). The resulting particles are consequently quality assured in terms of their stability (to the environment) and maximum deposition profiles (in the environment) in which they are to be eventually used.

There are a variety of engineering liquids and in preferred aspects of the second embodiments, these liquids enabled the same substance to be engineered with the a variety of attributes that suit the inhalation requirements of the patient. This has important implications as it enables the selection of particle attributes that maximises therapeutic effect whilst reducing the dose required for the therapeutic effect. Thus this invention emphasises the importance of bringing the engineering process more in line with the requirements of the end user to facilitate drug targeting and delivery in order to maximise therapeutic effect whilst minimising the side effects.

Hence it is more likely that these particles, when inspired, will be carried to deposit, in a similar fashion, in the targeted region(s) of a patient whose inhalation flow rate matches the inhalation gas flow rate used to engineer and test these particles.

It is clear that engineering particles using the temperature and environmental humidity conditions in line with that of the patient airways facilitated targeting of the engineered particles to the lower airways. Also, immersion of the feedstock in liquid nitrogen and/or moisturising the aerosol particles with the boiling vapour of the liquid nitrogen also facilitated targeting of the engineered particles to the lower airways. Additionally, engineering the particles at temperatures above body temperature facilitated targeting of the engineered particles to the lower airways.

The particles of the present invention can be engineered to maximally deposit the particles in the targeted regions of the patient airways at least at that patient inhalation flow rate. The morphological and/or topographical attributes dictated to the particles also enable the engineered particles to be suitable for a wider range of patient inhalation flow rates whilst still maximally depositing the particles in the targeted regions of the patient airways.

In another preferred aspect of the present invention, the inhalation gas flow rate can match the variation of the patient inhalation flow rate during the patient normal inspiratory profile.

The particles can be engineered at any inhalation flow rate even at inhalation flow rates much below that generated by patients with chronic or acute respiratory conditions. It is more likely that engineered particles of the present invention are more suitable for those patients with low inspiratory flow rates. Since the particles can be engineered at any specific flow rate, it is then possible to produce particles that match the inhalation flow rate of any patient or disease state so as to target specific patient categories and specific disease states. Additionally, the engineered particles can be used to achieve local and systemic effects irrespective of the physico-chemical properties of the substance (for example small molecules and macro-molecules, hydrophobic, hydrophilic and combinations thereof).

From this it is evident that particles can be engineered to match the inspiratory flow rate of any given patient. Equally particles can be engineered to match the resistance of the inhaler device. Subsequent, knowledge of the patient inspiratory flow rate and the resistance of the inhaler device enable particles to engineered by the process of this invention that compensates for insufficiencies in the patient inspiratory flow rate and the resistance of the inhaler device.

In a preferred aspect of the invention, one or more parts or all of the mimicked respiratory airway can be used to engineer and/or test the particles. In choosing the correct set of interconnected sections it is possible to engineer the particles such that they deposit highly and target the naso-pharynx, oropharynx, any part of the conducting zones of the airways, including the trachea, bronchi, bronchioles and terminal bronchioles or any locations within the respiratory zones of the airways including bronchioles, alveolar ducts or alveolar sacs and combinations thereof.

As described in the first embodiment, the mimicked respiratory system can be a twin-stage liquid impinger, an Andersen cascade impactor, although any other mimicked respiratory system or parts thereof can be used.

The engineered particles, formulations containing the engineered particles and compositions and/or inhaler devices containing the engineered particles should be suitably identified in the patient information leaflet and/or the inhaler device to indicate the inhalation flow rate used to engineer and test the particles and also indicating the patient inhalation flow rate which the engineered particles give maximum targeted deposition as well as indicating the regions of the lung targeted by these engineered particles at any inhalation flow rates. Hence, by measuring patient inhalation flow rate using devices such as peak flow meters, it is possible to give the patient the appropriate inhaler device containing engineered particles that maximally deposit at the target regions of the lung required by the patient at that patient's inhalation flow rate. This consequently enables easy identification by the patient or healthcare professional of the formulation compositions or inhaler device containing the foregoing that best suits the patient according to their patient inhalation flow rate.

### Third Embodiment

The process of using inhalation flow rate to engineer the particle can be facilitated by effervescence. Effervescent substances as used herein are substances that can comprise one or more substances which can act together or individually to evolve a gas. The gas released by the effervescence can be any gas, vapour or combinations thereof. Preferably effervescent gas(es) include but are not limited to Carbon dioxide, oxygen, nitrogen, nitric oxide and combinations thereof. The preferred gas is carbon dioxide.

In a preferred aspect, the starting feedstock can contain an acid-base couple which effervesce and release carbon dioxide, when brought into contact with water or water vapour present in the microenvironment, that is generated by the inhalation flow rate. The effervescence and consequent formation and release of bubbles, lowers the density of the aerosol particle and confers to the aerosol particle additional attributes that can assist the inhalation flow rate to engineer the particles with morphological, topographical and aerodynamic diameters dictated by that inhalation flow rate. Since the inhalation flow rate facilitates the transfer of the effervescence initiator and promoter (i.e. water vapour) to the aerosol particle it also removes the products of the effervescence. In removing the products of effervescence the aerosol particle is consequently lighter. Hence inhalation flow rate enhances effervescence of the particle and this effervescence enhances aerosol particle engineering.

The acid component of the couple can comprise one or more acids and the base component of the couple can comprise one or more bases. Preferable acid components include but are not limited to aliphatic carboxylic acids and mineral acids and acids present in the human body. Non-limiting examples of suitable acids include citric acid, tartaric acid, malic acid, fumaric acid, adipic acid, succinic acid, acid anhydrides of such acids and combinations thereof. Citric acid is a preferable acid.

Preferable base components includes but are not limited to alkali metal or alkaline earth metal carbonates, bicarbonates, sequi-carbonates and combinations thereof. Non-limiting examples of suitable bases include carbonates (for example calcium carbonate, sodium glycine carbonate, ammonium carbonate), bicarbonate (for example sodium bicarbonate, potassium bicarbonate) sequi-carbonates and mixtures thereof. Sodium bicarbonate is a suitable base.

In a preferred aspect the drug present in the aerosol particle can be an acid or base.

In a preferred aspect of the third embodiment, the acid and the base can be incorporated into the feedstock that forms the aerosol particle and the micro-environment around the aerosol particle contains the effervescence initiator and promoter (water or water vapour)

In another preferred aspect of the third embodiment, the base couple can be incorporated into the feedstock that forms the aerosol particle and the micro-environment around the aerosol particle contains the effervescent initiator and promoter as well as the corresponding acid couple.

In another preferred aspect of the third embodiment, the acid couple can be incorporated into the feedstock that forms the aerosol particle and the micro-environment around the aerosol particle contains the effervescent initiator and promoter as well as the corresponding base couple

In a preferred aspect of the third embodiment, effervescence of the aerosol particles occurring during particle flight will assist the inhalation flow rate to further engineer the aerosol particles with attributes and aerodynamic diameters favourable and dictated by the inhalation flow rate. In a further preferred aspect, effervescence can continue after particle impaction either by it's deposition in a liquid media that promotes effervescence or by the effect of inhalation flow rate bringing the micro-environment (containing the effervescence initiator and promoter) to the deposited particles.

Effervescent particles are extremely useful for delivery of drugs into the lungs. The bubbles formed by effervescence can incorporate one or more components present in the effervescencing particle. Bubbles are low in mass and inertia and are aerodynamically favourable and will be carried and directed by the inhalation flow rate to deep lung. Bubbles also have high gaseous contents that enable rapid diffusion of it's contents into and through difficult membranes and thick mucus (like that present in cystic fibrotics). Effervescent particles that deposit in the upper airways continue to generate bubbles that are carried and directed, by the inhalation flow rate, to the lower lung enabling the targeting to both upper and lower lung. Additionally the particles are made more aerodynamically favourable to the inhalation flow rate by effervescence, hence more particles should reach and deposit in the lower airways. Those particles that deposit in the lower airways will further effervesce in the lung fluids causing particle disintegration, increasing the specific surface area of the deposited particles from which there is improved drug dissolution and bioavailability. Consequently, effervescent particles are applicable to local and systemic drug delivery.

### EXAMPLES

I) Engineering Elegant Particles from Solid Feedstock:

A bulk solid feedstock material suitable for engineering was produced using the below outlined process.

5 g of α-lactose monohydrate (Borculo-Dormo, U.K) was dissolved in 100 ml distilled water and the resulting solution was spray-dried using a Buchi 190 mini-spray dryer according to the following conditions:
Inlet temperature: 176° C.,
Outlet temperature: 112° C.,
Aspirator dial reading: 15,
Lactose solution feed rate: 5 ml/min,

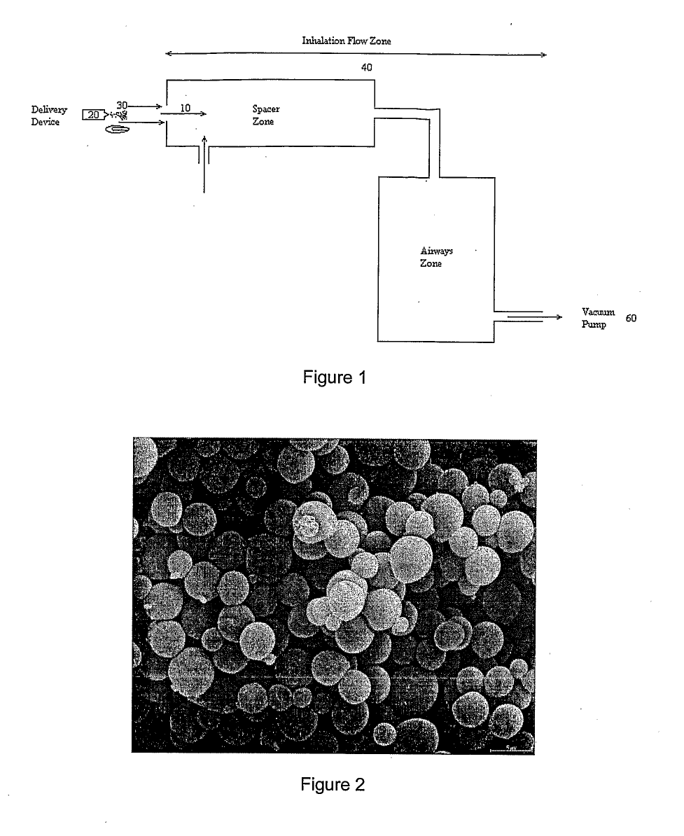

The spray dried lactose was collected and stored in a desiccator over silica gel until further use. The resulting particles are shown in FIG. 2

Note from FIG. 2, the particles of the starting feedstock are both spherical and smooth.

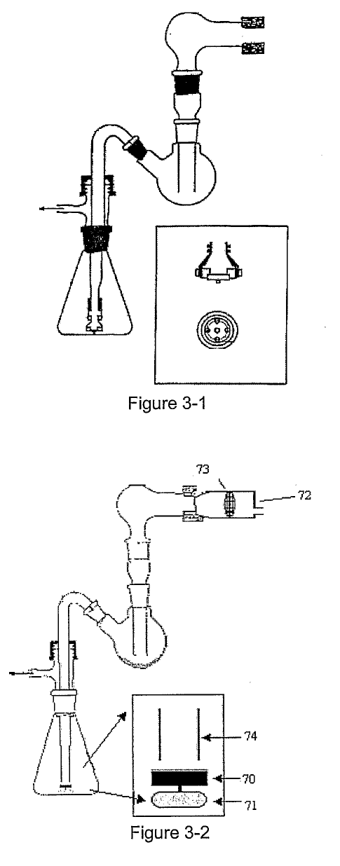

A twin stage impinger as described in Apparatus A of the British Pharmacopoeia, BP 2003 [Volume IV, Appendix XII FA255] (FIG. 3-1) is routinely used to measure the aerodynamic diameter of airborne particles and to assess the in-vitro deposition of inhaled particles. The twin stage impinger has two impaction stages, the upper stage (upper impingement chamber), a round bottomed flask, traps agglomerates and non-inhalable particles whilst the lower stage, a conical flask (lower impingement chamber), is aimed to trap all the smallest particles with aerodynamic diameter less than 6.4 μm. 7 ml of a liquid is normally placed in the upper stage (i.e. the round bottomed flask) to prevent bouncing of impacted particles. The cut off diameter of the upper stage at 60 L/min is 6.4 μm (Miller, N. C., Marple, V. A., Shultz, R. K. & Poon, W. S., Assessment of the twin impinger for size measurement of metered dose inhaler sprays. Pharm. Res., 1992; 9:1123-1127).

A modified twin stage impinger apparatus (mTSI) was developed for this invention (FIG. 3-2). Comparison of FIGS. 3-1 and 3-2 shows the modification brought to the original twin stage impinger. In the modified twin-stage impinger assembly, a microscope stub (70) was placed on a piece of blu-tak® (71) in the flask, directly in line with the coupling tube (74) from the upper stages of the impinger representing the lower stage of the twin stage impinger.

### Example 1

### Bulk Solid Feedstock, Delivery from a Breath Activated Device and Mimicked Respiratory System Containing Water

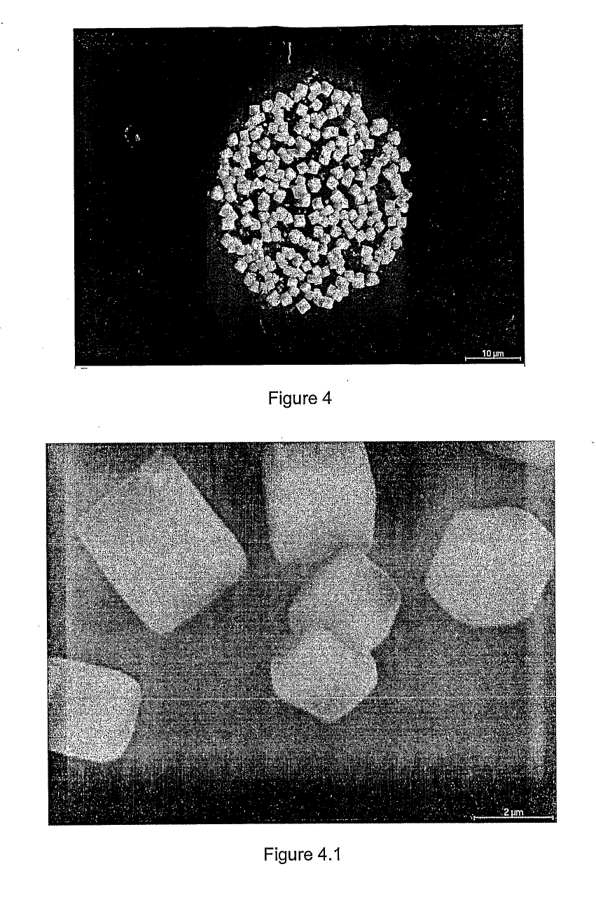

7 ml of distilled water was placed in the upper stage of the twin stage impinger whilst the lower stage was free of any liquid. A rotahaler (72) (GSK, Ware U.K.) was fitted into a moulded rubber mouthpiece attached to the throat piece of the impinger. The mouthpiece also having a filter(73) housed therein to ensure that oversize particles do not get into the system. Once the assembly had been checked and found to be airtight and vertical, the vacuum pump was switched on and the inhalation flow rate was adjusted at 60 L/min and 100 mg of spray dried lactose powder, as shown in FIG. 2, was introduced using a glass weighing boat into an orifice at the rear of the rotahaler device. Two seconds after all the spray dried lactose was drawn, the vacuum pump was switched off and the twin stage impinger dismantled to recover the microscope stub containing deposited powder which was analysed using a scanning electron microscope, the results of which are shown in FIGS. 4 and 4.1

From FIG. 4 and FIG. 4.1 there are important observations. The particles of the invention are elegant cubes that are smooth, monodisperse, are individual cubes that are separated from each other suggesting that these cubes are non-cohesive and that these cubes also have aerodynamic diameters less than 6.4 μm. The particles have been morphologically changed, i.e. their shape is changed without affecting their size. Despite the starting spheres and cubes having the same size, the surface area of the cubes are larger than that of corresponding spheres of the same size, hence the specific surface area of the resulting particles is also increased.

### Example 2

### Bulk Solid Feedstock, Delivery From a Breath Activated Device and Mimicked Respiratory System Containing 50:50 v/v Water:Ethanol Mixture

The same as example 1, except that 7 ml of a 50:50 v/v water:ethanol mixture was used in the upper round bottomed flask instead of 7 ml distilled water.

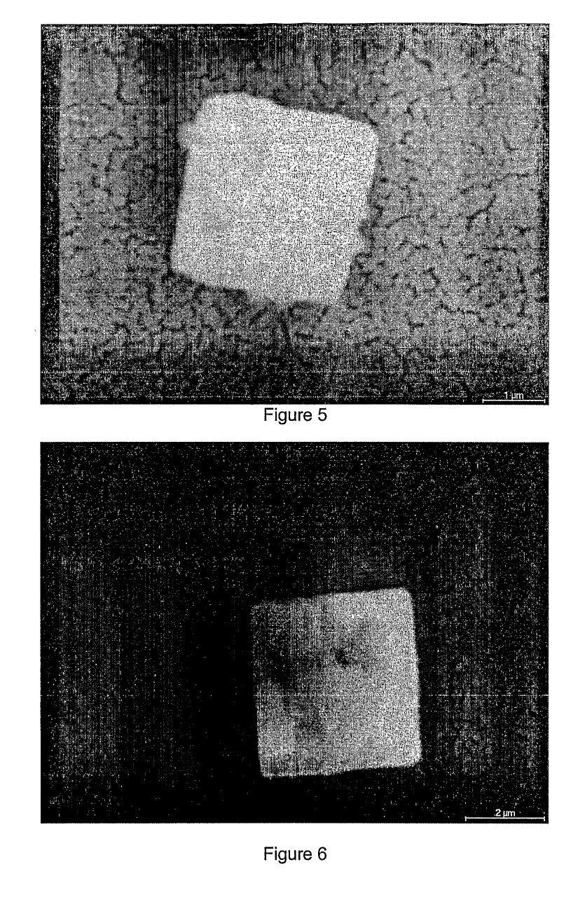

From FIG. 5, it is clear that cubes are also formed which are elegant cubes and have elements of surface roughness. The aerodynamic diameter of these engineered particles is less than 6.4 μm. This example shows that the inhalation flow rate can be used to change particle morphology and surface topography.

### Example 3

### Bulk Solid Feedstock, Delivery From a Breath Activated Device and Mimicked Respiratory System Containing Ethanol

The same as example 1, except that 7 ml of ethanol was used in the upper round bottomed flask instead of 7 ml distilled water

From FIG. 6, it is clear that cubes are again formed that are elegant and have increased elements of surface roughness. The aerodynamic diameters of these engineered particles are less than 6.4 μm. Note: Morphology changed and increased changes in topography. This example shows that cubic forms can be obtained using a non-solvent for the starting feedstock.

### Example 4

### Bulk Crystalline, Non-Cohesive, Rough Surfaced Solid Feedstock, Delivery From a Breath Activated Device and Mimicked Respiratory System Containing Water

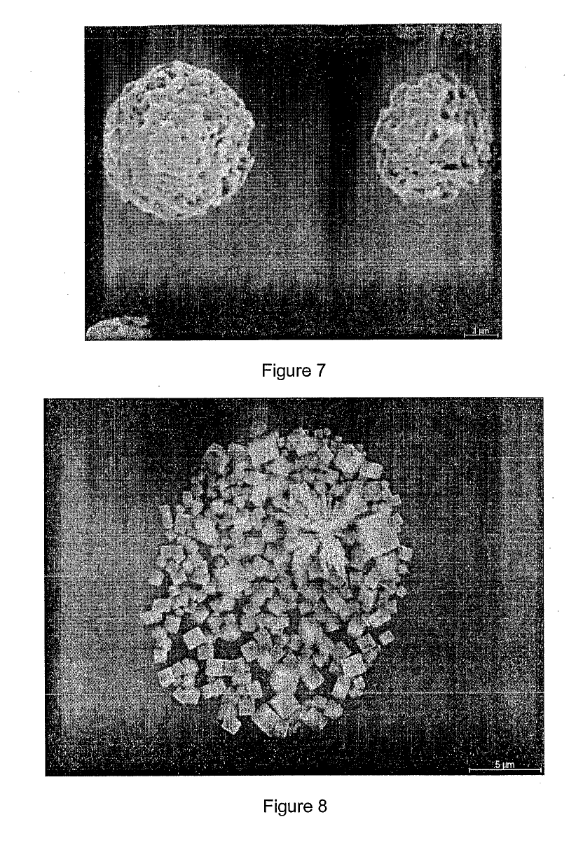

The experimental procedure was the same as example 1, except that the starting feedstock was spherical, crystalline, non-cohesive lactose that have rough surfaces as shown in FIG. 7.

From FIG. 8 it is clear that elegant cubes, which are smooth in surface, are formed. Amongst the particles are cubes and a few cuboidal crystals. Additionally the engineered cubes have aerodynamic diameter less than 6.4 μm.

### Example 5

### Bulk Solid Feedstock of Hydrophilic Therapeutic Agent, Delivery From a Breath Activated Device and Mimicked Respiratory System Containing Water

5 g of salbutamol sulphate was dissolved in 100 ml distilled water and the resulting solution was spray-dried in the same manner as described above. The resulting spray dried salbutamol sulphate particles were spherical, amorphous, smooth in surface and highly cohesive.

The spray dried salbutamol sulphate feedstock was engineered in a similar manner to that described in example 1.

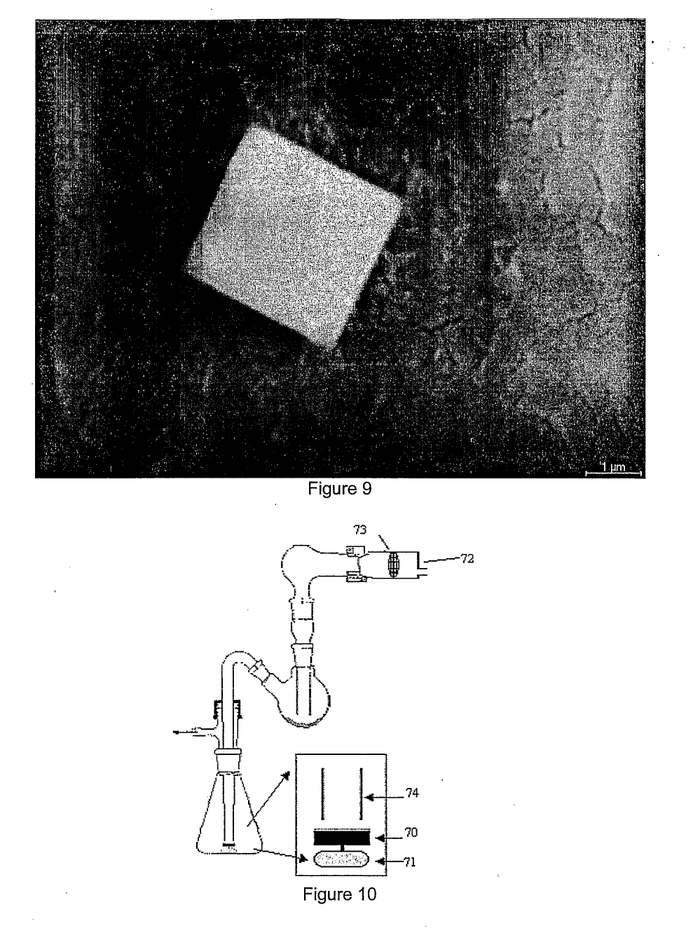

The particles obtained were elegant and cubic in shape, smooth in surface (FIG. 9). Note: Morphology changed.

II) Engineering Elegant Particles from Liquid Feedstock:

### Example 6

### Bulk Liquid Feedstock of Hydrophobic Therapeutic Agent, Delivery From a Self Actuated Device, Spacer Device, and a Mimicked Respiratory System

0.5 g Beclomethasone Dipropionate (BDP) was dissolved in 100 ml acetone to form a liquid feedstock of BDP solution. 10 ml of the BDP solution was loaded into the stainless steel cup of an Airbrush (model Simair XL 2000, Simair Graphics equipment Ltd, Harrogate). The BDP solution was atomised using air as the atomising gas at a pressure of 2 bars giving a BDP solution feeder rate of 11 ml/min.

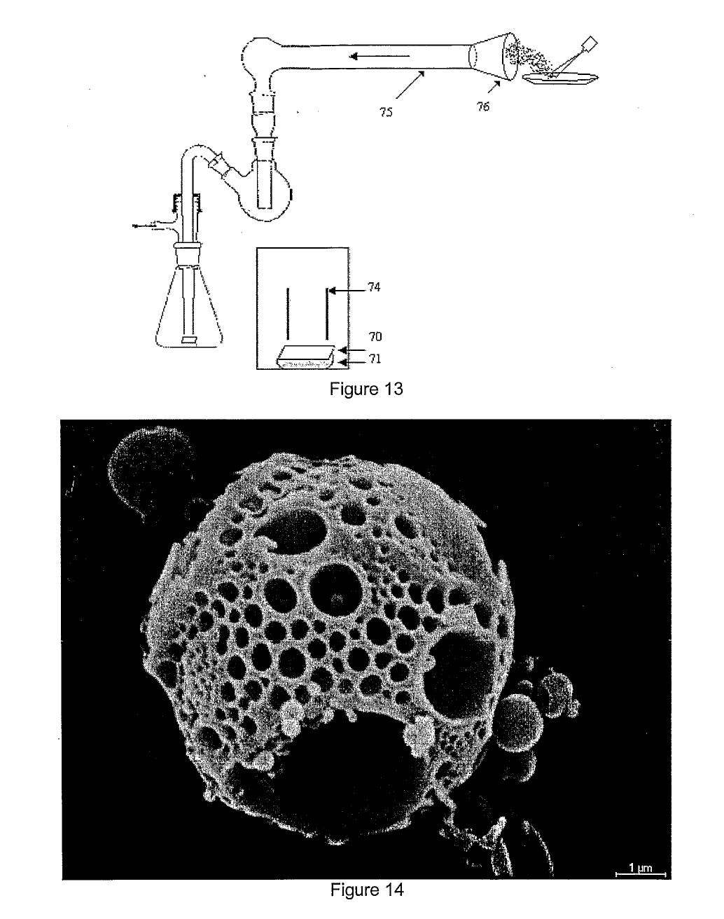

The atomised liquid feed stock was arranged such that the aerosol particles approached a liquid Nitrogen bed at an angle, not only to freeze the aerosol particles, cause expansion of the solvent (as described in patent WO 03059324) to expand and thus fracture the frozen aerosol particle forming freeze fractured aerosol particles which bounced off the surface of the liquid nitrogen to be intercepted by the inhalation gas flow such that the freeze fractured particles entered the inhalation flow zone. This inhalation flow zone comprised a spacer device (that is half a Volumatic®) connected to the moulded rubber mouthpiece of the mTSI throat, the latter being connected to the mTSI. The inhalation flow rate used to pull the frozen aerosol particles was 20 l/min. The resultant particles were recovered from the microscope cover slip in the upper flask of the mTSI. FIG. 13 shows a similar arrangement, except for the positioning of the microscope cover slip.

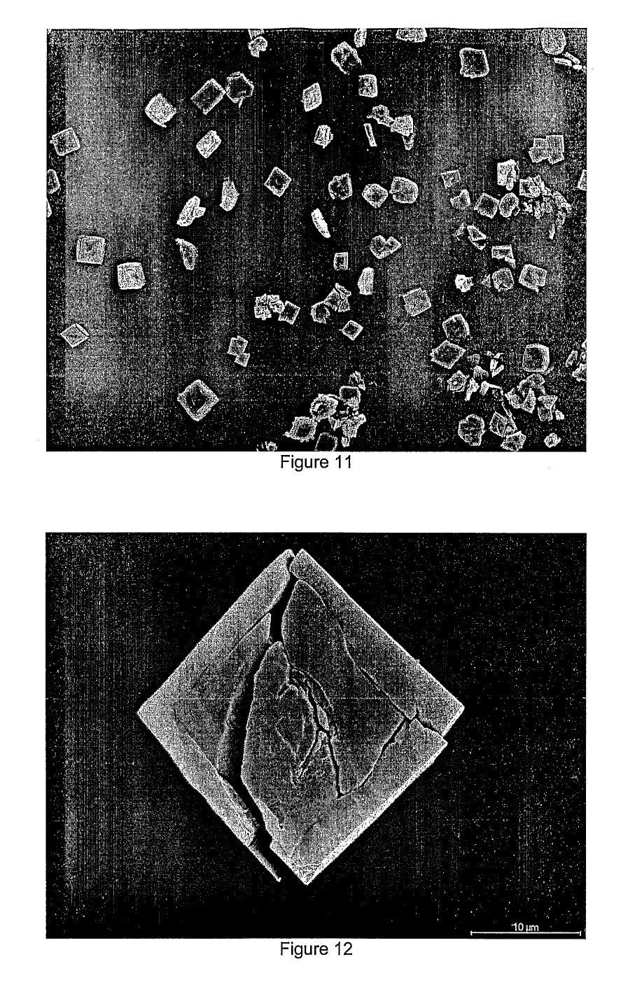

From FIGS. 11 and 12, the particles are not only cubic but also fractured further increasing the specific surface area of the particles and the particles are non-cohesive and separated from each other. Hence there have been changes in morphology and topography of the particles.

### Example 7

### Bulk Liquid Feedstock of Hydrophobic Therapeutic Agent, Delivery from a Self Actuated Device, with Two Interconnected Spacer Zones and Mimicked Respiratory System Containing No Liquids

The BDP solution from example 6 was atomised in a similar manner to example 6 except that there was an additional 50 cm, pyrex® spacer device(75) connected from one half of the volumatic®(76) and into the mouth piece of the mTSI throat and that the resultant particles were collected from the lower flask as shown in FIG. 13 as a result the aerosol particles travelled further hence there was an increased particle flight time.

The particle of FIG. 14 has a topography that may be described as craters, dents, depressions, entrances and indentations. These topographical attributes clearly increase the specific surface area of the particles.

From comparison of FIGS. 11, 12 and 14 it can be seen that the distance travelled by the aerosol particle and thus particle flight time enables further control, of at least, particle morphology (in terms of shape and size) and particle topography. Also the surface defects vary according to the flight time.

### Example 8

### Bulk Liquid Feedstock of Hydrophobic Therapeutic Agent, Delivery From a Self Actuated Device, Spacer Device Containing a Liquid That is Aspirated to Form a Vapour, Mimicked Respiratory System Containing No Liquids

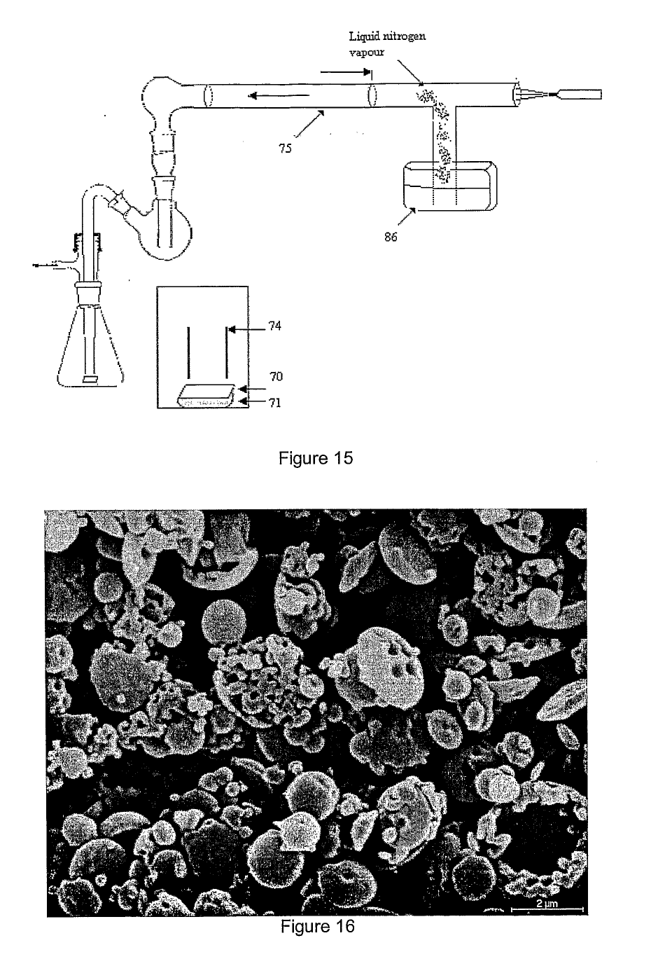

FIG. 15 diagrammatically depicts the experimental set-up used for this example. The inhalation flow zone comprised two interconnected spacer devices that are then connected to a modified mTSI. The first spacer device consisted of T-junction made of copper and had two inlet ports, one inlet port was used to introduce the feed and the other to introduce the vapour from liquid nitrogen. The bottom inlet port of the copper T-junction was placed in a stainless steel bowl(86) filled with liquid nitrogen thus freezing all the T-junction. The second spacer device connected to the T-junction was a 50 cm copper tubing. The other end of this 50 cm spacer was connected into the mouthpiece of the mTSI throat. The feed was a 1% solution of Beclomethasone dipropionate in acetone and 10 ml of the BDP solution was atomised (via the airbrush using air as the atomisation gas at two bar pressure) and supplied to the first spacer device. The inhalation gas flow rate of 60 L/min generated a stream of cold nitrogen vapour that was pulled into the spacer zone and thus into the mTSI as shown in FIG. 15.

In this example the particles were engineered at low temperature (below 25° C. i.e. below the boiling point of the solvent which in this case is acetone) in microenvironments generated within the spacer device of the inhalation flow zone.

### Example 9

### Bulk Liquid Feedstock of Hydrophobic Therapeutic Agent, Delivery From a Self Actuated Device, Spacer Zone Containing No Liquids, Mimicked Respiratory System Containing a Liquid

Using a similar schematic as in FIG. 15 except that 7 ml of acetone was placed in the upper stage of the mTSI. The Beclomethasone dipropionate (BDP) solution from example 8 was atomised such that the resultant aerosol was directed towards and entered the spacer of FIG. 15 at an inhalation flow rate of 60 L/min.

The liquid in the mimicked respiratory system (i.e. the upper flask of the mTSI) dynamically humidified the inhaled gas(es) creating microenvironments of acetone vapour around the aerosol particles leading to the formation of BDP particles with morphology and topography that is different from the above. Hence, it is clear that the type of liquid and the point of introduction of the liquid affect the morphology, or the topography or both. Additionally, for the same substance a variety of morphologies and topographies can be obtained.

### Example 10

### Effect of Inhalation Flow Rate on the Morphology and Topography of the Particles from Bulk Liquid Feedstock of a Hydrophobic Therapeutic Agent, Delivery From a Self Actuated Device Using a Spacer Zone and a Mimicked Respiratory System

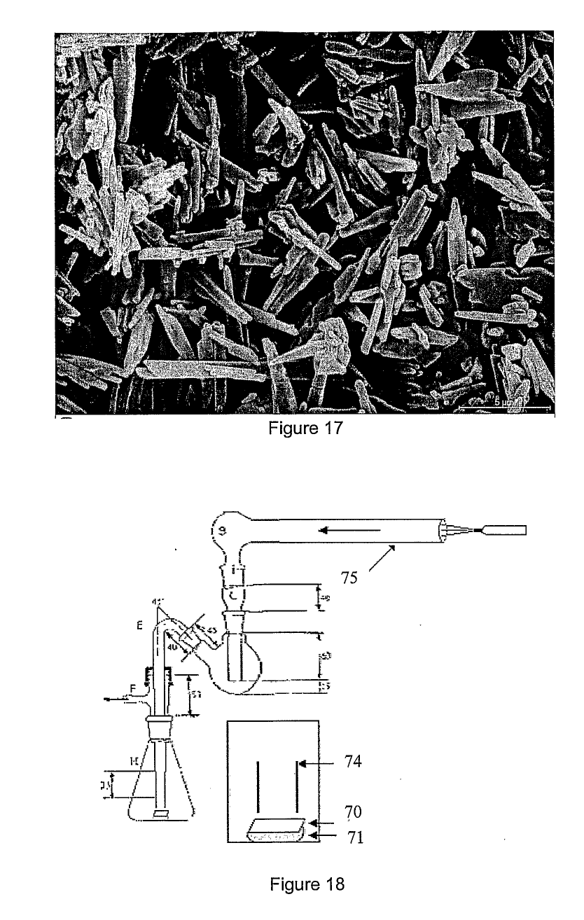

0.5 g Fluticasone Propionate (FP) was dissolved in 50 ml acetone to form a liquid feedstock of FP solution. 10 ml of the FP solution was atomised by an airbrush at a pressure of 2 bars to form an aerosol that was directed to enter into the mTSI via the 50 cm glass spacer, as shown in FIG. 18. There was no liquid present in either the spacer or mimicked respiratory system (mTSI).

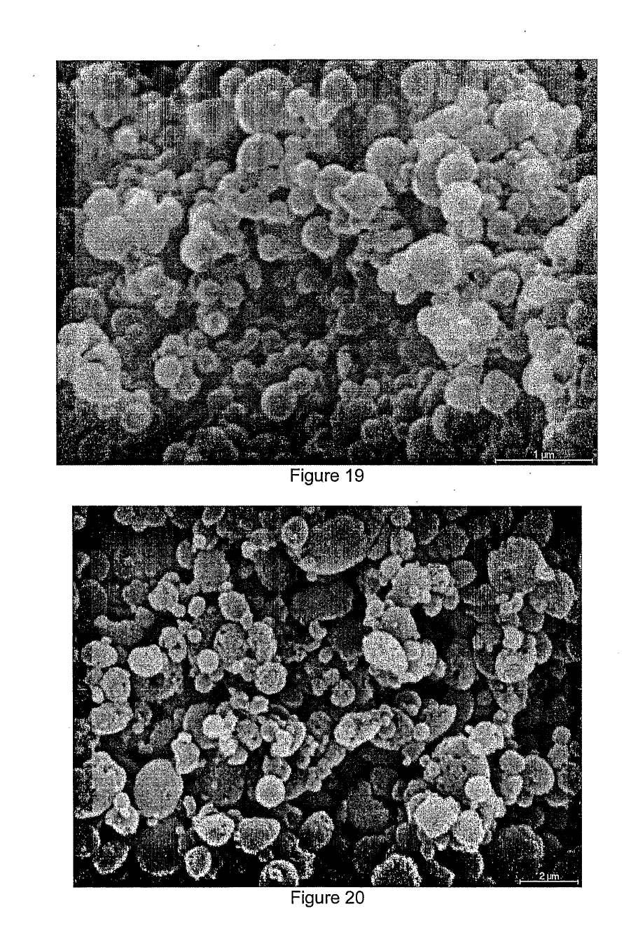

Three experiments were conducted one in which the inhalation gas flow rate in the inhalation flow zone is 0 L/min (i.e. NO INHALATION GAS FLOW), at 60 L/min and 120 L/min and the corresponding Figures for the engineered particles are FIGS. 19, 20 and 21 respectively, for the particles recovered from the lower stage of the mTSI.

The particles recovered at ZERO INHALATION FLOW RATE were highly cohesive, agglomerated and were concentrated in a very small area of the slide. The recovery of the particles was very poor. As can be seen from the scanning electron microscope the particles have smooth topography and spherical shape.

From FIG. 20, it is clear that the particles obtained at 60 L/min are spheroidal and have a rougher topography compared to those obtained at NO INHALATION FLOW RATE (i.e. FIG. 19). At 120 L/min the particles (FIG. 21) exhibited greater deviations from sphericity compared to 60 L/min and were rougher in surface topography. Additionally there is improved recovery of the particles when inhalation flow rate was used. Since the particles of FIGS. 20 and 21, all have aerodynamic diameters less than 6.4 μm, they are then targeted for deep lung delivery.

### Example 11

### Effect of Inhalation Flow Rate on the Morphology and Topography of the Particles From Bulk Liquid Feedstock of a Hydrophobic Therapeutic Agent, Delivery From a Self Actuated Device Using an Andersen Cascade Impactor as a Mimicked Respiratory System, Containing no Liquid(s) and a Spacer Zone Containing No Liquids

1: Air compressor connected to the air brush
2: Air Brush inserted in inlet port of pyrex tube
3: 50 cm pyrex tube
4: Glass throat of twin stage impinger
5: Andersen Cascade impactor
6: Vacuum pump

In order to more closely mimic the human airway, an Andersen cascade impactor was used.

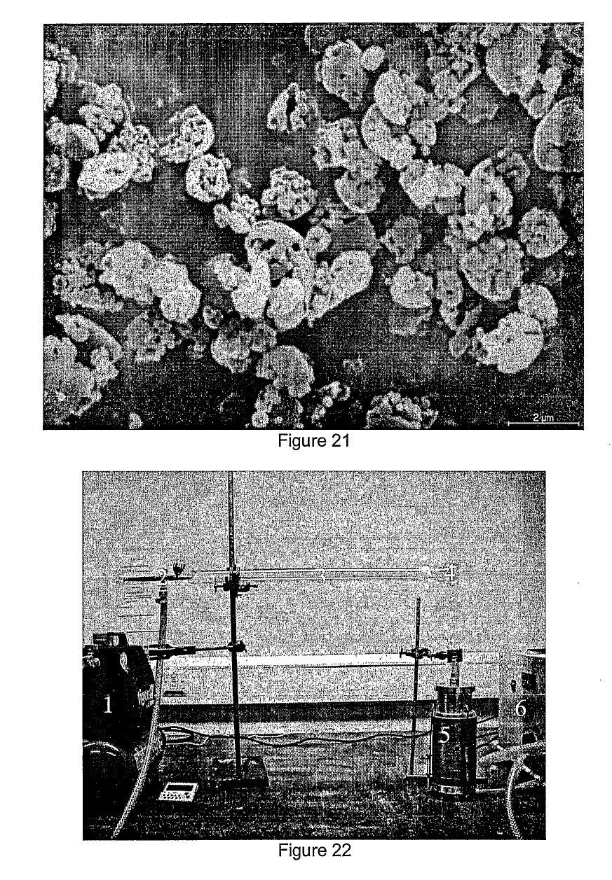

0.5 g of Fluticasone propionate (FP) was dissolved in 50 ml acetone. Once the assembly as indicated in FIG. 22 had been checked and found to be vertical and airtight, the vacuum pump was switched on to generate an inhalation flow rate of 28.3 L/min. 10 ml of the FP solution was loaded into the stainless steel cup of the Air brush. The atomisation head of the air brush was introduced into the inlet port of the pyrex tube and compressed air at a pressure of 2 bars was utilised to atomise the FP solution at a feed rate of 11 ml/min at ambient temperature. After atomisation the Andersen cascade impactor was dismantled and the engineered particles were recovered. The experiment was repeated using an inhalation flow rate of 60 L/min.

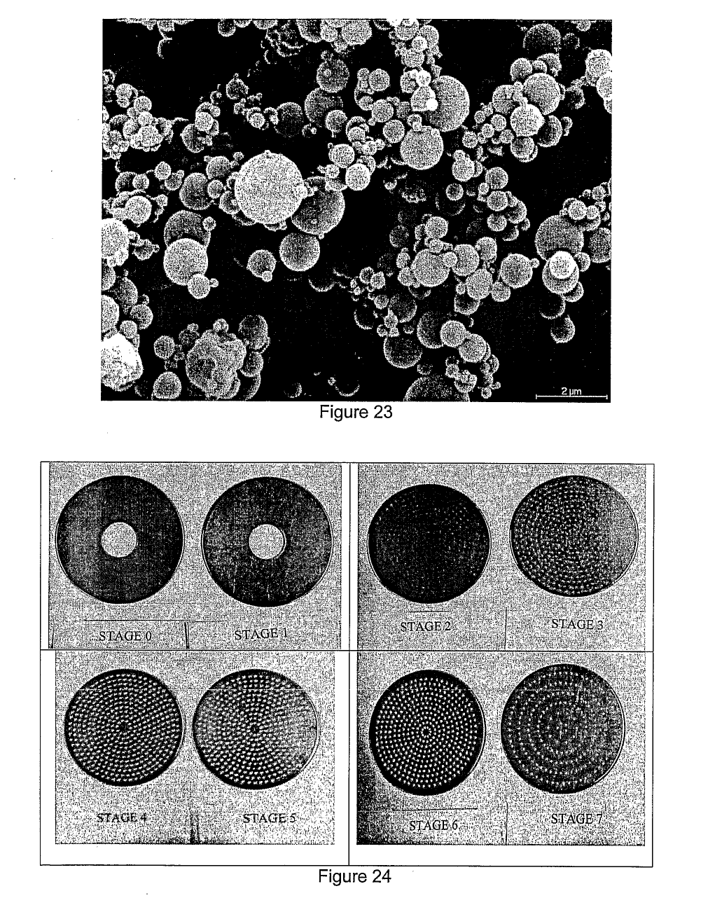

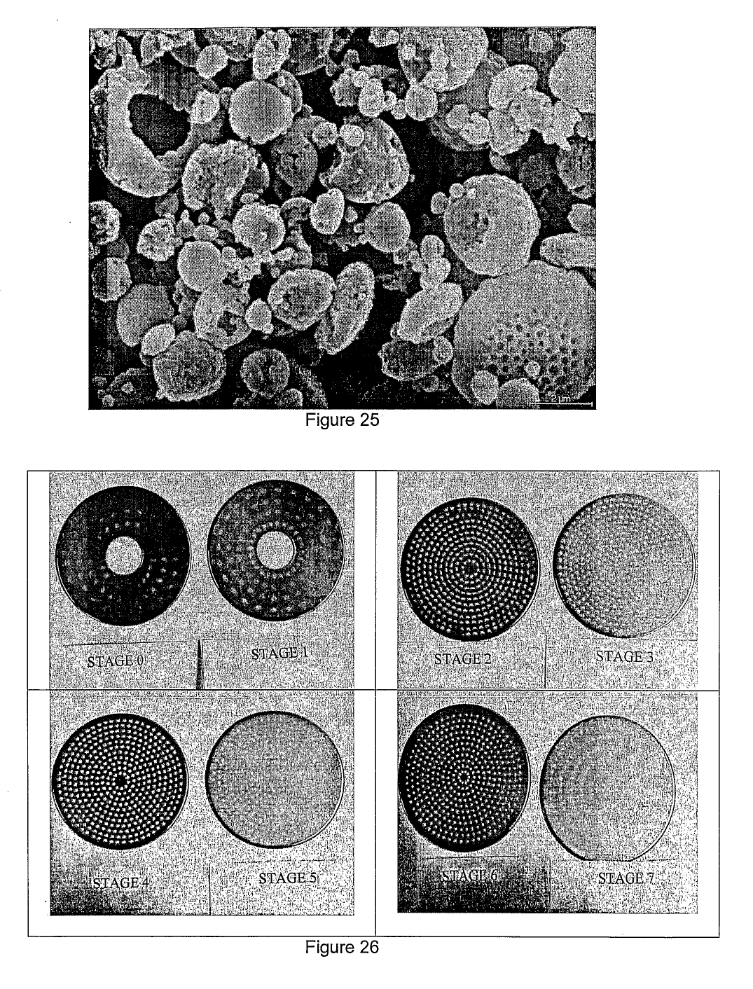

From FIGS. 23 and 25, it is clear that the inhalation flow rate not only dictates the morphology and topography of the particles but also their resulting aerodynamic diameters (FIGS. 24 and 26). The Fluticasone propionate (FP) particles engineered at a flow rate of 28.3 L/min (FIG. 24) are smooth and spherical whereas the same particles engineered at a flow rate of 60 L/min have a plurality of shapes. From the Andersen cascade impactor the FP particles engineered at 28.3 L/min deposited from stages 2 to 7 (FIG. 24), that have aerodynamic diameters of 0.4 μm to 5.8 μm corresponding to deposition from the pharynx to the terminal alveoli whilst the FP particles engineered at 60 L/min deposited from stage 0 to stage 7 (FIG. 26), corresponding to aerodynamic diameters of 0.4 μm-5.8 μm which represents deposition from the pharynx to the terminal alveoli and this is the same deposition region obtained with particles engineered at both inhalation flow rates at ambient temperature. Hence the engineered FP particles can be targeted for both upper and lower airways at either inhalation flow rates.

The lowest impaction stages of the Andersen cascade impactor enables the collection of particles with aerodynamic diameter of between 0.4 μm to 0.7 μm thus enabling engineering of particles to an accuracy of 0.3 μm in the aerodynamic diameter of the particles.

Note: inhalation flow rate affects the morphology and topography of the particles irrespective of the mimicked respiratory system in this case either twin stage impinger (Example 10) or Andersen cascade impactor (Example 11) hence using inhalation flow rate to engineer particles in a mimicked respiratory system is viable, consequently any other mimicked respiratory system or part thereof can be used.

### Example 12

### Effect of Inhalation Flow Rate on the Morphology and Topography of the Particles From Bulk Liquid Feedstock of a Hydrophobic Therapeutic Agent, Delivery From a Self Actuated Device Using an Andersen Cascade Impactor as a Mimicked Respiratory System, Containing Water and a Spacer Zone Containing No Liquid(s) at Body Temperature

This experiment was carried out using the same protocols as Example 11, except, to further mimic the environment representing the human respiratory airway humidity, 5 ml of water was placed in the pre-separator of the Andersen cascade impactor so that the inhalation gas flow would vaporise this water and humidify the Andersen cascade impactor. Additionally, to create an environment representing human respiratory airway temperature, the inhalation flow zone (i.e. items 3,4 and 5 of FIG. 22) were wrapped in a electrically heated blanket, maintained at 45° C., such that the inhalation gas flow through the inhalation flow zone, as indicated by a temperature probe was about body temperature.

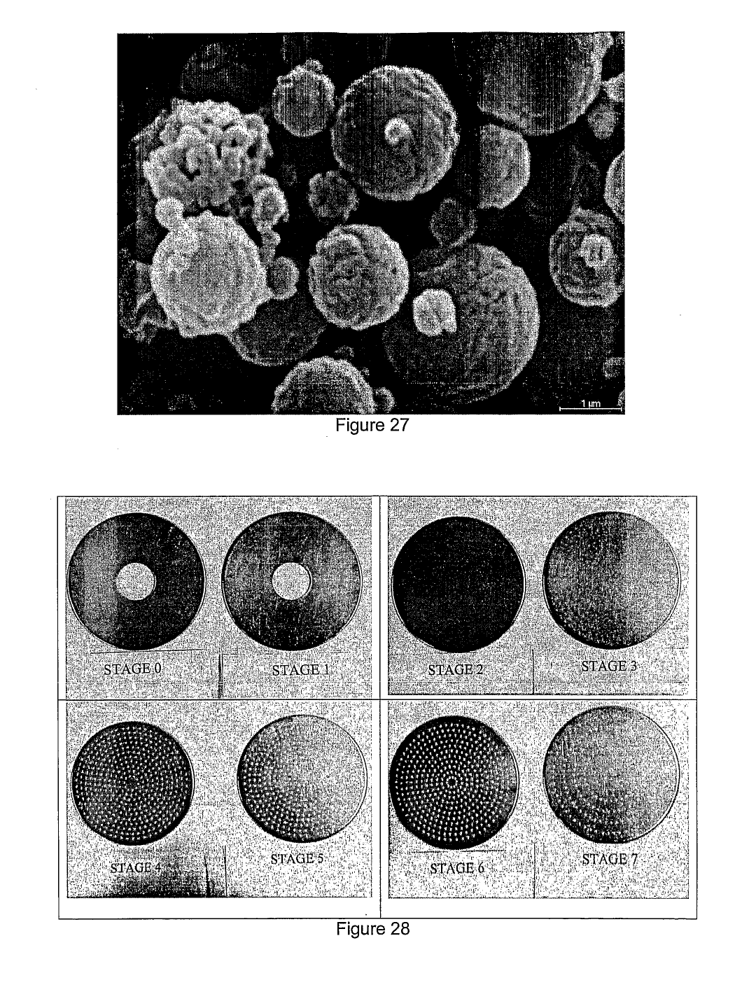

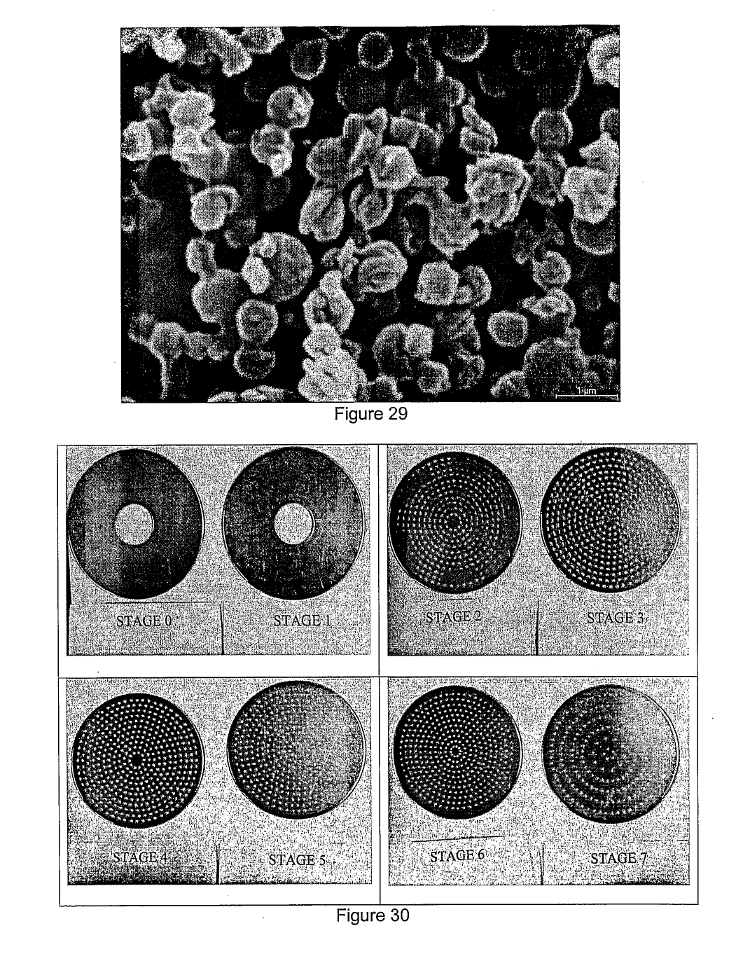

From FIG. 28, FP particles engineered under the temperature and humidity conditions present in the airways at 28.3 L/min, deposited from stage 3 to stage 7 of the Andersen cascade impactor; corresponding to aerodynamic diameters of 0.4 μm to 4.7 μm, representing deposition from trachea and primary bronchi to the terminal alveoli. Whilst those particles engineered under the temperature and humidity conditions present in the airways, at 60 L/min, deposited from stage 2 to stage 7 of the Andersen cascade impactor, corresponding to aerodynamic diameters of 0.4 μm-3.3 μm From FIGS. 27 and 29 it is clear that the topography and morphology of the particles engineered under conditions of temperature and humidity in the airways are different from that produced at the same inhalation flow rate in the absence of the temperature and humidity conditions in the airways (i.e. FIGS. 23 and 25, respectively).

Additionally the particles engineered in the temperature and humidity conditions present in the airways, reduces the upper limit of the aerodynamic diameter thus enabling targeting and maximised deposition of the engineered particles to the lower airways.

As these particles are engineered in an environment that is similar to that of the end user it is more likely that these particles would be more stable, and deposit in the same regions when inhaled by the end-user (i.e. the patient).

From the examples above it should be clear that, using the same substance, it is possible to engineer particles with a wide variety of morphologies and topographies whist assuring targeting and maximal deposition in the desired regions.

### Example 13

### Effect of Inhalation Flow Rate on the Morphology and Topography of the Particles From Bulk Liquid Feedstock of a Hydrophobic Therapeutic Agent, Delivery From a Self Actuated Device Using an Andersen Cascade Impactor as a Mimicked Respiratory System, Containing No Liquid(s) and a Heated Spacer Device Containing No Liquid(s) at Body Temperature

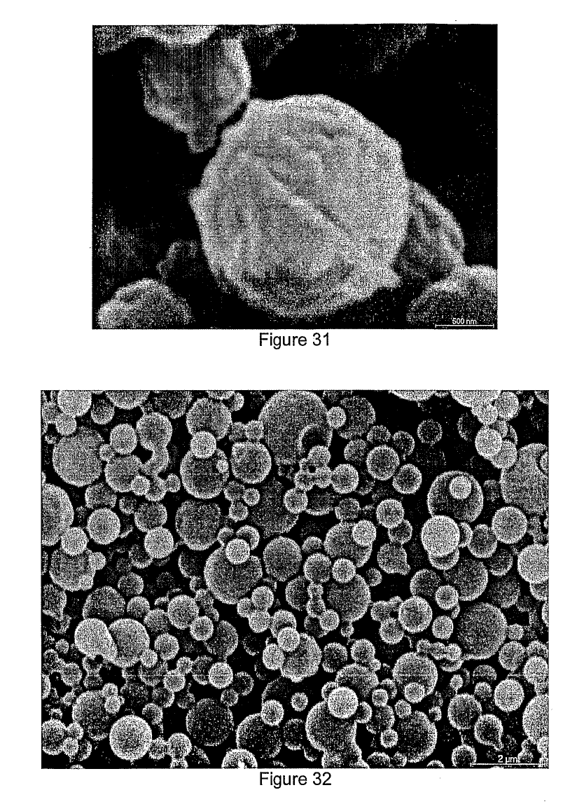

Using the same experimental protocols as Example 12, except that the pre-separator of the Andersen cascade impactor was liquid free Comparing FIG. 31 with FIG. 23, it is clear that the temperature dictates the formation of additional topographical and morphological attributes which can be considered as “fingerprints” representative of the effect of temperature during engineering, such fingerprints are not limited to the ridges seen in FIG. 31.

The deposition of the particles on the plates of the Andersen cascade impactor indicated targeting and maximised deposition of the engineered particles to the lower airways. Additionally, the use of temperature when engineering the particles reduces the upper limit of the aerodynamic diameter. Hence the temperature at which the particles were engineered influences the deposition of the resulting particles.

### Example 14

### Using of Inhalation Flow Rate to Engineer From Bulk Liquid Feedstock of a Hydrophilic Therapeutic Agent Delivered From a Self Actuated Device Using an Andersen Cascade Impactor as a Mimicked Respiratory System, Containing No Liquid(s) and a Heated Spacer Zone Containing No Liquid(s) at 45° C.

Using the same experimental procedures as example 13, except that the delivery device (item 2, FIG. 22) was an airjet nebuliser operated at a pressure of 1 bar to deliver (at 0.5 ml/min) a solution (prepared by dissolving 0.5 g Salbutamol sulphate in 50 ml water), as a low velocity aerosol to the inhalation gas flow of 60 L/min.

From FIG. 32 it is clear that a substance dissolved in water can be engineered by the inhalation flow rate at temperatures well below the boiling point of the water. Consequently, the inhalation flow rate enables engineering of particles from high boiling point liquids at temperatures much below the boiling point of the liquid.

### Example 15

### Bulk Liquid Feedstock of a Hydrophilic Therapeutic Agent in a Mixture of Liquids Delivered From a Self Actuated Device Using an Andersen Cascade Impactor at Inhalation Flow Rate of 60 L/min at 45° C.

Using the same experimental procedures as example 14, except that the solution was prepared by dissolving 0.1 g Salbutamol sulphate in 50 ml of a 80:20 v/v ratio of ethanol/water. The morphology of the particles collected from the Andersen cascade impactor was similar to that of FIG. 32.

### Example 16

### Bulk Liquid Feedstock of a Hydrophilic Therapeutic Agent in Water Delivered From a Self Actuated Device Using the mTSI at an Inhalation Flow Rate of 120 L/min at 45° C.

A 1% solution of salbutamol sulphate in water was nebulised using an airjet nebuliser operated at a pressure of 1.0 bar to deliver the feed (at 0.5 ml/minute) as a low velocity aerosol into an inhalation flow zone consisting of a spacer and mTSI according to the assembly shown in FIG. 18. The inhalation flow zone was maintained at a temperature of 45° C. by wrapping an electrically heated blanket around the inhalation flow zone. The inhalation flow rate used was 120 L/min.

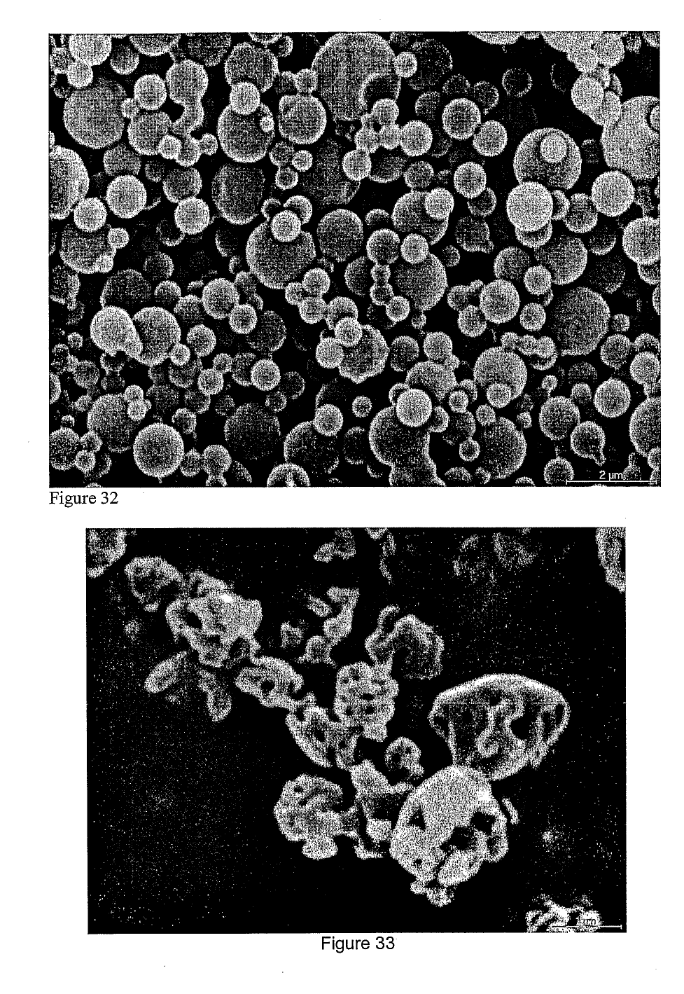

From FIGS. 32 and 33 it is clear that the inhalation flow rate dictated the morphology and topography of the engineered particles even from a non-volatile and high boiling point solvent. The morphology and topography of the particles engineered at a flow rate of 120 L/min is such that they have increased specific surface area compared to those engineered at 60 L/min.

### Example 17

### Bulk Liquid Feedstock of a Hydrophilic Therapeutic Agent Dissolved in Water Delivered From a Self Actuated Device Using the mTSI at Inhalation Flow Rate of 120 L/min Using Pre-Heated Inhalation Gas

The feedstock of a 1% solution of Salbutamol sulphate in water was nebulised using an airjet nebuliser (78) operated at a pressure of 1.0 bar to deliver the feedstock (at 0.5 ml/minute) as a low velocity aerosol into an inhalation flow zone consisting of a two copper spacer devices and mTSI according to the assembly shown in FIG. 34. The aerosol entered one inlet port of the first spacer device while the other inlet was used to introduce inhalation gases that were heated by means of a hair drier(77). The inhalation flow rate used was 120 L/min.

The morphology and topography of the resulting salbutamol sulphate particles were similar to that depicted in FIG. 33.

### Example 18

### Bulk Liquid Feedstock of a Protein Dissolved in Water Delivered From a Self Actuated Device Using the mTSI at Inhalation Flow Rate of 120 L/min at 45° C.

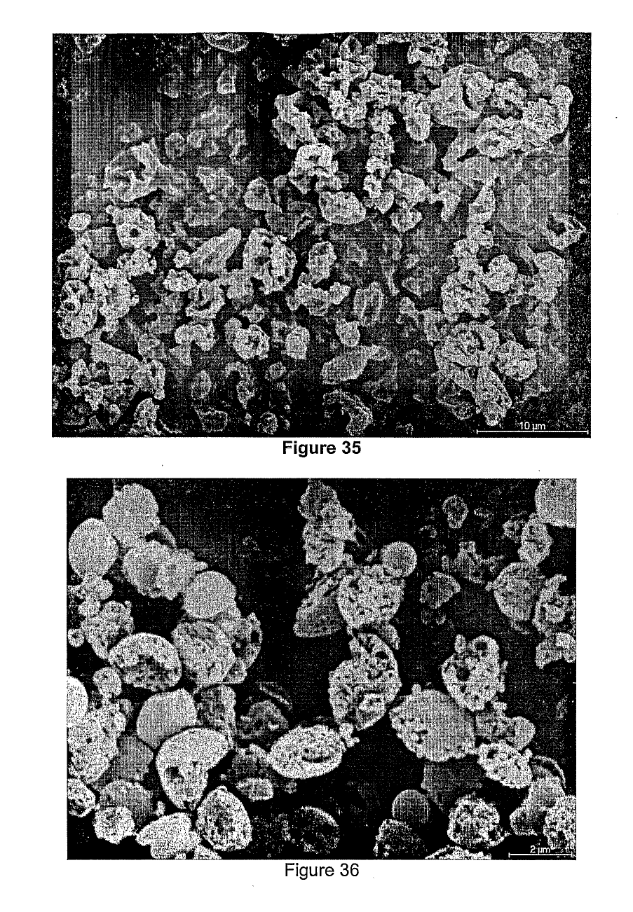

Using the same experimental procedures as example 16, except that the solution was prepared by dissolving 0.1 g Bovine Serum Albumin (BSA) in 10mi water and the resulting feed was nebulised from an airjet nebuliser operated at a pressure of 1.0 bar to deliver the feedstock (at 0.5 ml/minute) as a low velocity aerosol into an inhalation flow zone that consisted of a 50 cm copper tube connected to a mTSI according to the assembly shown in FIG. 18. The inhalation flow zone was maintained at a temperature of 45° C. by wrapping an electrically heated blanket around the inhalation flow zone. The inhalation flow rate used was 120 L/min. The particles obtained are shown in FIG. 35.

It is clear that the process is suitable for engineering protein substances hence it is also suitable for other delicate biological macro-molecules such as genes.

### Example 19

### Bulk Liquid Feedstock Containing a Hydrophobic Therapeutic Agent, Delivered From a Self-Actuated Device, Spacer Device and Modified Twin Stage Impinger

0.5 g of Beclomethasone dipropionate was dissolved in 100 ml of acetone, 10 ml of the resulting solution was atomised using an airbrush (whose atomisation protocols are described in Example 10), at ambient temperature (i.e. below 25° C.), to form a high velocity aerosol that enters the inhalation flow zone (as depicted in FIG. 18). 7 ml of ethanol was placed in the upper flask of the twin stage impinger and the inhalation flow rate was 60 L/min. The resulting engineered BDP particles are shown in FIG. 36.

### Example 20

### Bulk Liquid Feedstock Containing a Hydrophobic Therapeutic Agent, Delivered From a Self-Actuated Device, Spacer Device and Modified Twin Stage Impinger

0.5 g of Budesonide was dissolved in 100 ml of acetone, 10 ml of the resulting solution was atomised using an airbrush (whose atomisation protocols are described in Example 10), at ambient temperature (i.e. below 25° C.), to form a high velocity aerosol that entered the inhalation flow zone (as depicted in FIG. 18). No liquid was placed in the upper flask of the twin stage impinger and the inhalation flow rate was 60 L/min.

### Example 21

### Bulk Liquid Feedstock Containing Two Hydrophobic Therapeutic Agents, Delivered From a Self-Actuated Device, Spacer Device and Modified Twin Stage Impinger

The experimental procedure and conditions used were the same as example 19, except that the starting feedstock was a solution of a 1% mixture of Fluticasone propionate (FP) and salmeterol xinafoate (SML) in acetone. The ratios of Fluticasone propionate to salmeterol xinafoate employed in the mixtures were 10 to 1, 5 to 1 and 2 to 1 w/w, respectively.

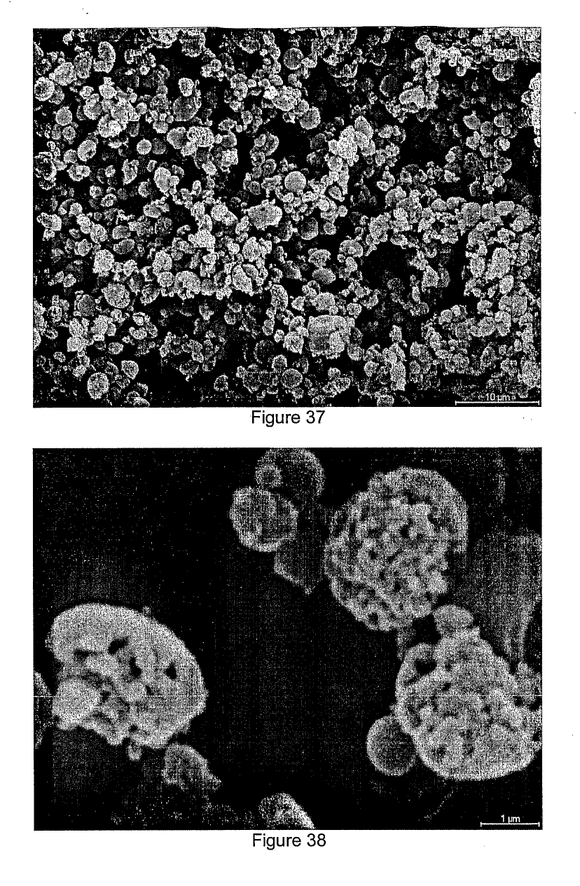

FIG. 37 is a general view of the engineered particles obtained with the 10 to 1 ratio of Fluticasone propionate to salmeterol xinafoate, whilst FIG. 38 is a close view of these same particles.

### Example 22

### Bulk Liquid Feedstock Containing Two Therapeutic Agents One Hydrophobic and One Hydrophilic, Delivered From a Self-Actuated Device, Spacer Zone and Modified Twin Stage Impinger

A 1% solution of Beclomethasone dipropionate[BDP]/Salbutamol sulphate[SS] (in the ratio 50 to 50 w/w) in a 50 ml of an acetone/water mixture (in the ratio 80 to 20 v/v) was prepared by dissolving 0.25 g of BDP in 40 ml acetone to form a solution that was mixed with 10 ml of water containing 0.25 gm dissolved SS. The resulting solution was atomised using an airbrush (whose atomisation protocols are described in Example 10) into an inhalation flow zone as depicted in FIG. 18. The particles were engineered at ambient temperature using an inhalation flow rate of 120 L/min.

Similarly, a 1% solution of Beclomethasone dipropionate [BDP]/Salbutamol sulphate[SS] in the ratio 80 to 20 w/w was prepared by combining 40 ml of acetone containing 0.4 g BDP and 10 ml of water containing 0.1 gm of SS was engineered in a similar manner.

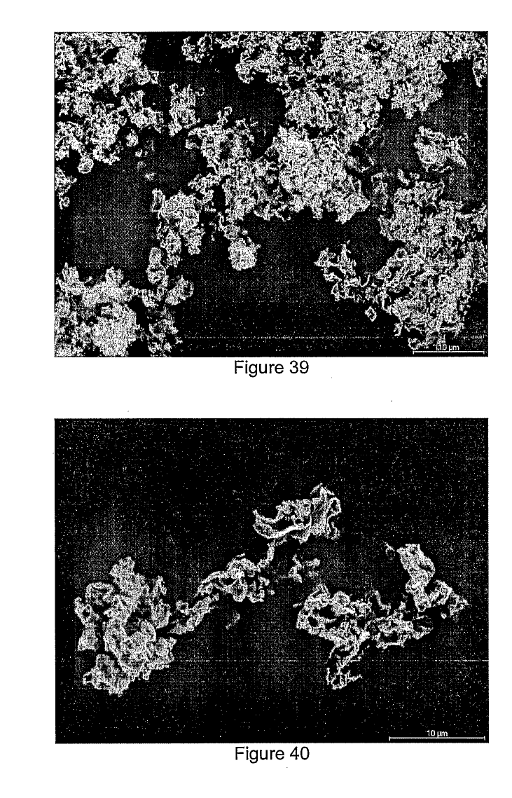

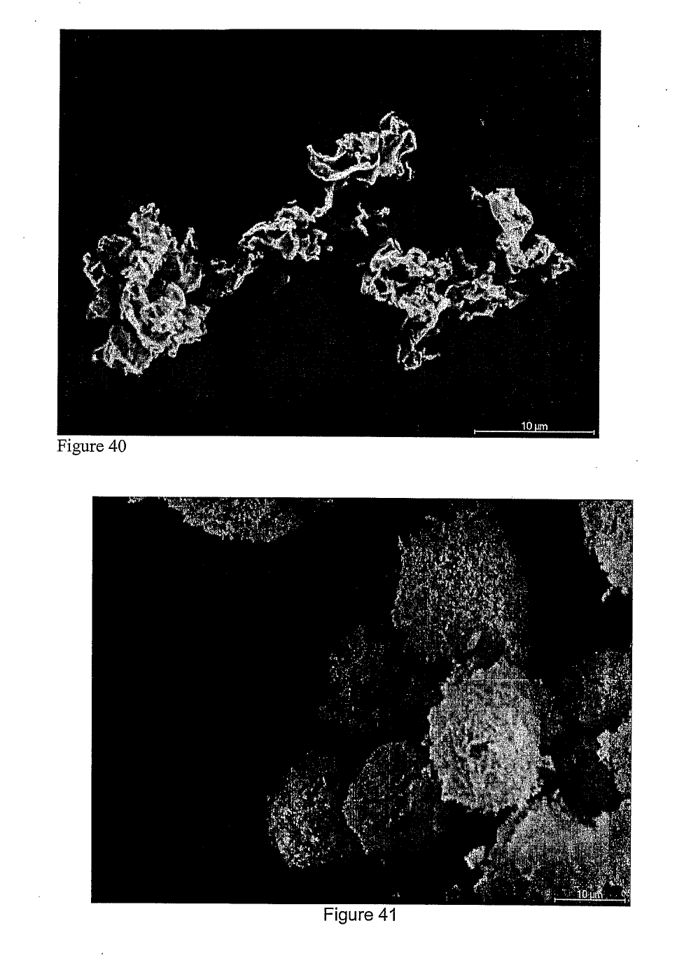

The particles of FIGS. 39 and 40 are irregular in shape and do not have a defined geometric form and they also exhibited high specific surface area.

From examples 21 and 22 it is clear that inhalation flow rate can be used to engineer and produce combination drug particles, these combinations may be hydrophobic-hydrophobic, hydrophobic-hydrophilic and even hydrophilic-hydrophilic combinations. Since two substances were incorporated into one particle it is possible to use the inhalation gas flow rate to engineer particles containing more than two substances.

### Example 23

### Use of Inhalation Flow Rate to Engineer Particles After Deposition of the Particles in a Mimicked Respiratory System That is a Modified Twin Stage Impinger

20 ml of ethanol(80) was placed in the lower flask of the Twin Stage Impinger whilst the upper flask was liquid free (FIG. 42). 500 mg of spray-dried lactose(81) (as obtained above) was placed in a glass weighing boat(79) and introduced into a Quickfit device (which had a grid) that was connected to the throat of the mTSI. The inspiratory gas flow rate was adjusted to 20 L/min and the Quickfit device facilitated de-aggregation, dispersion and aerosolisation of the spray-dried lactose. The aspiration flow rate was maintained for two minutes after all the spray dried powder had been aspirated.

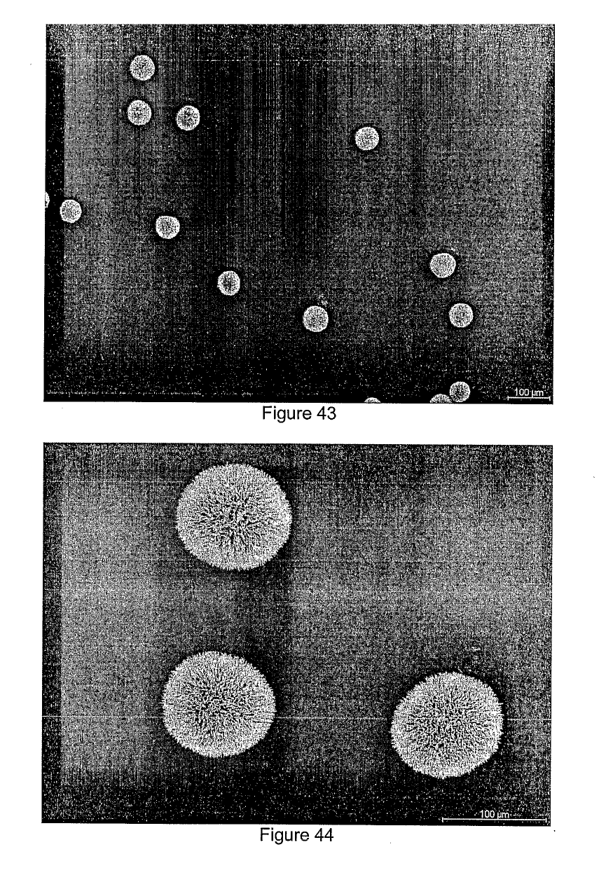

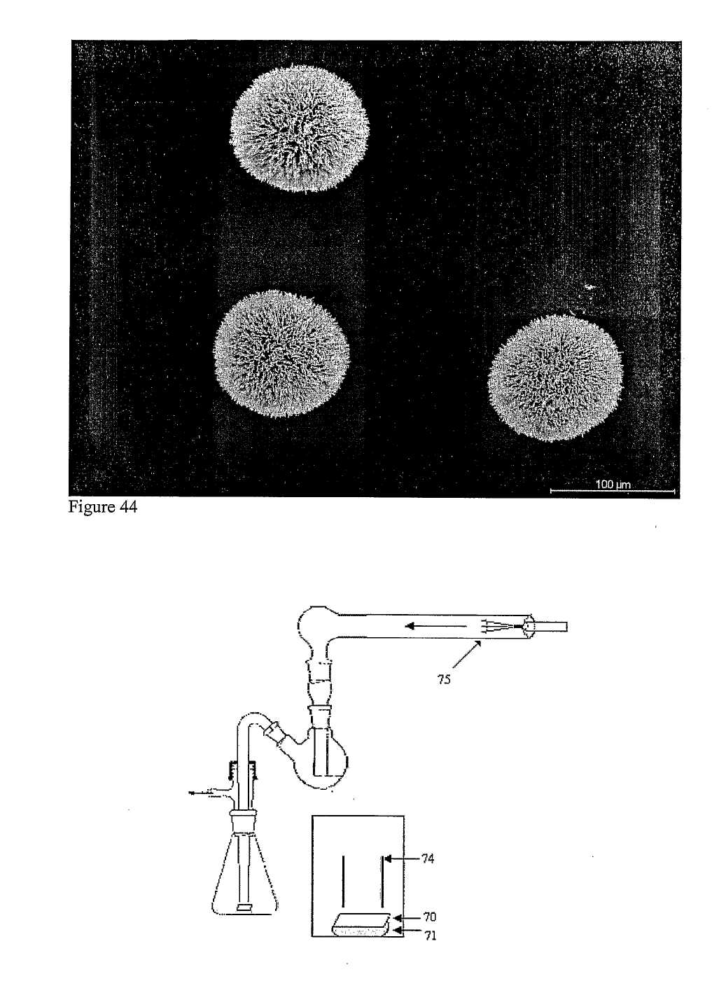

The suspension of lactose particles in ethanol recovered from the lower flask of the Twin Stage Impinger was poured above a 0.45 μm membrane filter (Whatman, Maidstone U.K) and the remaining ethanol in the liquid was removed from the particles by suction. The resulting engineered particles are shown in FIGS. 43 and 44.

From FIGS. 43 and 44 the particles have increased in size compared to the original spray-dried material (as shown in FIG. 2), the particles are monodisperse, uniform in size shape and have “hairs”. Since the particles have grown in size this suggest that they are more crystalline than the starting material.

### Example 24

### Bulk Liquid Feedstock Containing a Hydrophobic Therapeutic Agent, Delivered From a Breath-Actuated Device, Spacer Zone and Modified Twin Stage Impinger

0.5 g of Fluticasone propionate (FP) was dissolved in 100 ml of acetone, 10 ml of the resulting FP solution was loaded into the stainless steel cup of an Air brush(82) (model SIMair XL2000 Simair Graphics Equipment Ltd, Harrogate). The atomisation head of the air brush was inserted into the inlet port of a spacer device (50 cm glass tube) connected to a modified twin stage impinger containing a microscope slide in its lower flask. According to this assembly as shown in FIG. 45, there is no gap between the delivery device and the spacer device. After switching the aspiration pump on the FP solution was sucked through the airbrush from the reservoir in the stainless steel cup, thus atomising the FP solution into the spacer device. The inhalation flow rate used was 150 l/min and the inhalation power generated therefrom resulted in a feed rate of 7.5 ml/mn. The engineered particles are shown in FIG. 46.

### Example 25

### A Method of Scaling Up the Inhalation Flow Rate Engineering Process

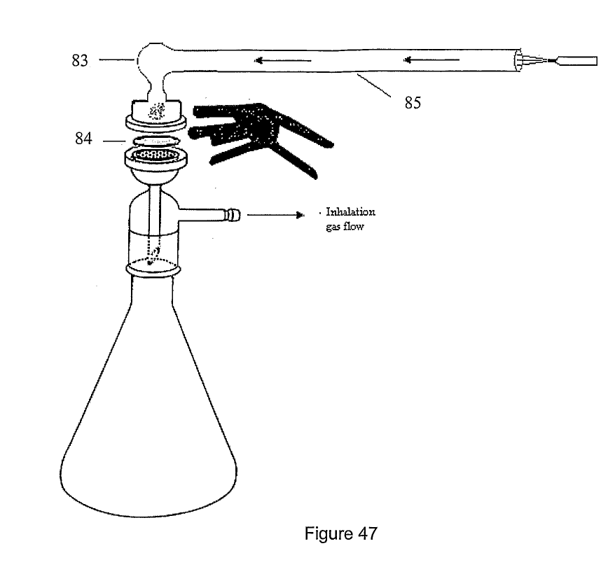

10 g Beclomethasone Dipropionate (BDP) was dissolved in 1 L of acetone. The inhalation flow zone comprised a 100 cm tubular spacer device(85) connected to the glass throat of a twin stage impinger, this glass throat(83) was connected to a modified filtration unit(84) (comprising a suction flask and the filter holder with a whatman filter paper, size No 1) adapted to fit the glass throat (FIG. 47). The inhalation flow rate was established at 20 L/min and the BDP solution was atomised using an airbrush operating at 2 bar pressure, at ambient temperature into the spacer device. The glass throat was used to mimic part of the respiratory system and the filtration unit facilitated collection of the particles. This scheme was successfully used to engineer and collect the resulting particles at many inhalation gas flow rates using many materials.

### Example 26

### A Method of Scaling Up the Inhalation Flow Rate Engineering Process Using a Vacuum Cleaner

The inhalation flow zone consisted of a 100 cm copper tube connected to the flexible tubing of a vacuum cleaner (Sonic, model C2001S, 1300 watts). The vacuum cleaner was switched on and the BDP solution of Example 23 was atomised into the copper tube. The inhalation gas flow rate generated by the vacuum cleaner was used to generate the inhalation gas flow rate to engineer and collect the particles in the filter bag of the vacuum cleaner. The flexible tubing of the vacuum cleaner was also connected onto the USP throat.

Another vacuum cleaner used to generate the inhalation flow rate to engineer and collect the particles was Tyson® DC07 cyclone vacuum cleaner which can collect particles as small as 0.1 μm making such vacuum cleaners suitable for engineering, collecting inhalable particles.

### Example 27

Using the schematic, as shown in FIG. 42, except that the ethanol in lower stage of the mTSI was replaced by a microscope slide secured to the lower flask by blu-tak. The inhalation gas flow rate through the inhalation flow zone was adjusted to 120 L/min. Some liquid nitrogen was added to 0.5 g of micronised salbutamol sulphate and the resulting suspension was poured into the glass weighing boat of FIG. 42. The amount of powder recovered from the lower stage of the mTSI was determined. The experiment was repeated with 0.5 g of micronised salbutamol sulphate that was not immersed in liquid nitrogen. The quantity of powder recovered from the lower stage of the mTSI, using liquid nitrogen, was substantially greater than that without. Hence immersion of the feedstock in liquid nitrogen facilitates targeting and maximal deposition of the particles.

Using the schematic, as shown in FIG. 15, except that the atomiser of FIG. 15 was replaced by the glass weighing boat of FIG. 42. The inhalation gas flow rate through the inhalation flow zone was adjusted to 120 L/min and 0.5 g micronised salbutamol sulphate was introduced into the glass weighing boat. After aerosolisation the engineered particles were collected in the lower stage of the mTSI. Aspiration of the liquid nitrogen in the holding chamber to meet the micronised salbutamol sulphate increased the amount of salbutamol sulphate recovered in the lower stages of the mTSI thus improving targeting whilst maximising deposition of salbutamol sulphate.

### Example 28

### Engineering Particles in a Low Pressure Environment

100 mg of spray dried lactose powder (as shown in FIG. 2) was placed into the rear end of a rotahaler device. 7 ml of ethanol was placed in the upper flask of the mTSI as shown in FIG. 3-2. The entire assembly as shown in FIG. 3-2 (containing both the spray dried lactose and ethanol) and the vacuum pump (that generates the inhalation flow rate) were placed into a sealed BIG NEAT fume cabinet. The extractor fan of the fume cabinet was switched on consequently reducing the pressure within the fume cabinet to about 0.8 bar. The vacuum pump was switched on to generate an inhalation flow rate of 60 L/min. This inhalation flow rate pulled the spray dried lactose feedstock into the mTSI to enable the liquid within the mTSI to engineer the resulting aerosol particles. This example was repeated except that the extractor fan of the BIG NEAT fume cabinet was switched off and a pump was used to introduce air into the cabinet so as to raise the pressure within the fume cabinet to about 1.1 bar. This example illustrates the use of inhaled gas which is at pressures below and above that of atmospheric pressure to engineer and produce particles.

### Example 29

### Preparation of an Aerosol That Effervesces During Aerosol Particle Flight to Form Particles With Attributes That Make the Resulting Particles Suitable for Inhalation

The principle for this example is derived from effervescent tablets technology which is applied to aerosol science by initiating a reaction between an acid-base couple that is citric acid/sodium bicarbonate, respectively. The reaction between the couple triggers the evolution of carbon dioxide that is used in combination with the inhalation flow rate to engineer the aerosol particles.

50 mg of sodium bicarbonate was dissolved in 20 ml of distilled water whilst 0.1 g of Fluticasone propionate (FP) was dissolved in 80 ml of acetone. The resulting solutions were mixed to form a solution of Fluticasone propionate/sodium bicarbonate. 3.5 g of anhydrous citric acid was dissolved in 7 ml of distilled water, this citric acid solution was placed into the upper flask of the mTSI as shown in FIG. 18.

The inhalation flow rate was adjusted to 60 l/min. 10 ml of the FP/sodium bicarbonate solution was atomised using an airbrush at a pressure of 2 bars, at room temperature, through the inhalation flow zone to form an aerosol.

The inhalation flow rate promoted vaporisation of the citric acid solution to form microenvironments rich in water vapour (that contains citric acid) around the FP/sodium bicarbonate aerosol particles. The microenvironments contained sufficient water (such that when the inhalation flow transfer the water onto the aerosol particles) triggers the reaction between the acid-base couple (citric acid/sodium carbonate) resulting in the evolution of carbon dioxide, within the particle which further contributes to the engineering of the aerosol particles.

1-35. (canceled)

36. A method of producing particles from a feedstock material that comprises at least one therapeutic substance, prophylactic substance, diagnostic substance and/or excipient, for the use in the delivery of drugs to a selected drug delivery target region by inhalation, whereby at least one particle attribute, selected from the group consisting of morphology, topography or aerodynamic diameter, is engineered to provide particles with the aerodynamics necessary to become deposited at the selected drug delivery target region of a patient type with a known inhalation power, said method comprising the steps of:
a) providing a mimicked respiratory system, which is adapted to simulate at least one of the drug delivery target regions of the mammalian respiratory system;
b) providing an engineering medium within the mimicked respiratory system at a controlled temperature to create an environment that is conducive to the production of engineered particles, said medium comprising at least one gas;
c) operating the mimicked respiratory system to simulate a controlled inhalation flow rate within the system for a set period of time, whereby the controlled inhalation power imparted on the mimicked respiratory system is equal to or greater than the inhalation power of the known patient type;
d) providing an aerosolized feedstock material within the mimicked respiratory system, whereby the type of engineered particles produced in the reaction of the feedstock material with the engineering medium is dictated by the controlled inhalation power;
e) collecting the resultant engineered particles from a simulated drug delivery target region, provided by the mimicked respiratory system, that corresponds to the selected drug delivery target region.

37. The method of claim 36, wherein the mimicked respiratory system is provided by a modified twin stage impinger, an Andersen cascade impactor, or any other device capable of simulating at least one drug delivery target region of the mammalian respiratory system.

38. The method of claim 36, wherein the drug delivery target regions simulated by the mimicked respiratory system are selected from the group consisting of naso-pharynx; oropharynx; trachea; bronchi; bronchioles; alveolar ducts; and alveolar sacs.

39. The method of claim 36, wherein the inhalation flow rate within the mimicked respiratory system is between 1 and 1000 L/min.

40. The method of claim 36, wherein the inhalation flow rate within the mimicked respiratory system is set at a rate which simulates a natural inhalation flow rate of a mammalian lung, which is between 15 and 120 L/min.

41. The method of claim 36, wherein the feedstock material comprises at least one of the following constituents:
i) a therapeutic, prophylactic, or diagnostic substance;
ii) a liquid;
iii) an excipient; and
iv) a base and/or an acid.

42. The method of claim 36, wherein the feedstock material is sprayed into the mimicked respiratory system to provide the aerosolized feedstock material within the mimicked respiratory system.

43. The method of claim 36, wherein the feedstock material is sucked into the mimicked respiratory system to provide the aerosolized feedstock material within the mimicked respiratory system.

44. The method of claim 43, wherein the inhalation flow rate within the mimicked respiratory system provides the suction to draw the feedstock material into the mimicked respiratory system.

45. The method of claim 36, wherein the feedstock material comprises at least one therapeutic substance selected from the group consisting of corticosteroids; anti-inflammatories; anti-tussives; bronchodilators; and proteins.

46. The method of claim 36, wherein the feedstock material comprises at least one therapeutic substance selected from the group consisting of beclomethasone diproprionate; budesonide; fluticasone propionate; salmeterol xinofoate; salbutamol sulphate; and bovine serum albumin.

47. The method of claim 36, wherein the feedstock material comprises at least one excipient selected from the group consisting of monosaccharides; disaccharides; polysaccharides; and sugar alcohols.

48. The method of claim 36, wherein the feedstock material is passed through a filter before entering the mimicked respiratory system to control the size of the aerosolized feedstock material particles.

49. The method of claim 48, wherein the feedstock material is filtered to permit only particles having a diameter of 100 μm or less to enter the mimicked respiratory system.

50. The method of claim 36, comprising the further step of pre-treating the feedstock before it is introduced into the mimicked respiratory system.

51. The method of claim 50, wherein the pre-treatment step involves subjecting the feedstock to at least one liquefied gas.

52. The method of claim 36, wherein the engineering medium further comprises at least one fluid selected from the group consisting of water; a ketone; an alcohol; a fluorocarbon; a fluoroalkane; an acid; a base; a liquefied gas; and combinations thereof.

53. The method of claim 52, wherein the at least one fluid is selected from the group consisting of water; acetone; ethanol; hydrofluoroalkanes; chlorofluorocarbons; and liquid nitrogen.

54. The method of claim 52, wherein the step of providing an environment within the mimicked respiratory system further comprises agitating the engineering medium by directing the controlled inhalation flow through the at least one liquid present in the mimicked respiratory system.

55. The method of claim 36 wherein the inhalation flow gas is selected from the group consisting of air; nitrogen; oxygen; carbon dioxide; helium; argon; and combinations thereof.

56. The method of claim 36, wherein the temperature in the mimicked respiratory system is controlled at a temperature of between −200 and 200° C.

57. The method of claim 36, wherein the temperature is maintained at between −50 and 120° C.

58. The method of claim 36, wherein the temperature is maintained at a level which simulates that of the mammalian lungs, which is between 34 and 42° C.

59. The method of claim 36, wherein the engineering medium contains at least one effervescent substance.

60. The method of any of claim 36, wherein the feedstock material contains at least one effervescent substance.

61. The method of any of claim 59, wherein carbon dioxide is evolved by the combination of a base and an acid in an effervescent reaction.

62. The method of claim 36, wherein the mimicked respiratory system further comprises at least one spacer device.

63. The method of claim 62, wherein the at least one spacer device is provided in the mimicked respiratory system at a point between where the feedstock material is introduced into the mimicked respiratory system and the point where at least one of the simulated drug delivery target regions are provided by the mimicked respiratory system.

64. The method of claim 62, further comprising the step of creating a local environment within each spacer device that is distinct from that within the rest of the mimicked respiratory system.

65. The method of claim 62, wherein each provided spacer device comprises at least one inlet and at least one outlet, whereby the introduction of an engineering medium is used to control the internal environment of each spacer device.

66. The method of claim 36, further comprising the step of analyzing the particles deposited at the one or more simulated drug delivery target regions provided by the mimicked respiratory system, and using the results collected to provide feedback on a particular particle engineering environment.
